(19) United States
(12) Patent Application Publication
(10) Pub. No.: US 2024/0041782 A1
(43) Pub. Date: Feb. 8, 2024

(54) Oral Capsule Cannabinoid Formulations

(71) Applicants: Michael OGBURN, Lake Forest, CA (US); Christopher PRICE, Monroe Township, NJ (US)

(72) Inventors: Michael OGBURN, Lake Forest, CA (US); Christopher PRICE, Monroe Township, NJ (US)

(21) Appl. No.: 18/265,464

(22) PCT Filed: Dec. 10, 2021
(86) PCT No.: PCT/US2021/062784

§ 371 Date: Jun. 6, 2023

Related U.S. Application Data
(60) Provisional application No. 63/231,391, filed on Aug. 10, 2021.
(60) Provisional application No. 63/131,657, filed on Dec. 29, 2020.
(60) Provisional application No. 63/124,257, filed on Dec. 11, 2020.

(51) Int. Cl. A61K 9/50; A61K 31/00; A61K 9/48; A61K 9/00

(52) U.S. Cl. CPC A61K 9/5084; A61K 31/658; A61K 9/4875; A61K 9/0053; A61K 9/5047

(57) ABSTRACT

Provided herein are pharmaceutical or dietary supplement compositions, kits comprising pharmaceutical or dietary supplement compositions, methods of treating and preventing disease, and methods of making compositions and kits described herein. The pharmaceutical or dietary supplement composition compositions described herein can be administered orally. The pharmaceutical or dietary supplement compositions can be administered by a capsule described herein.

### CROSS REFERENCE TO RELATED APPLICATIONS

This application claims the benefit of U.S. Provisional Application No. 63/124,257, filed Dec. 11, 2020, U.S. Provisional Application No. 63/131,657, filed Dec. 29, 2020, and U.S. Provisional Application No. 63/231,391 filed Aug. 10, 2021, the disclosures of which are incorporated herein by reference in their entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are pharmaceutical compositions and dietary supplement compositions, in unit dose form, comprising a first active ingredient or a pharmaceutically acceptable salt thereof, comprising a plurality of spray dried particles, each particle of the plurality of spray dried particles comprising a cannabinoid or a pharmaceutically acceptable salt thereof, substantially encapsulated in a coating material. In some embodiments, within the plurality of spray dried particles comprising the cannabinoid, or the pharmaceutically acceptable salt thereof, substantially encapsulated in the coating material, individually can have a particle diameter ranging from about 50 micrometers to about 150 micrometers, as measured by a particle analyzer using laser diffraction. In some embodiments, the coating material can comprise a hydroxypropyl methylcellulose (HPMC), a hydroxypropyl methylcellulose acetate succinate (HPMCAS), a maltodextrin, a povidone, a copovidone or any combination thereof. In some embodiments, the plurality of spray dried particles can be at least partially surrounded by a first capsule, a second capsule, or both. In some embodiments, the first capsule can be surrounded by the second capsule. In some embodiments, the pharmaceutical or dietary supplement compositions described herein can further comprise a second active ingredient, or a pharmaceutically acceptable salt thereof in unit dose form. In some embodiments, the pharmaceutical or dietary supplement compositions described herein can further comprise an excipient. In some embodiments, the pharmaceutical or dietary supplement compositions described herein can comprise a second active ingredient that is at least partially surrounded by the first capsule, the second capsule or both. In some embodiments, the pharmaceutical or dietary supplement compositions described herein can comprise a second active ingredient that is at least partially surrounded by the first capsule. In some embodiments, the pharmaceutical or dietary supplement compositions described herein can comprise a second active ingredient that is at least partially surrounded by the second capsule. In some embodiments, the pharmaceutical or dietary supplement compositions described herein can comprise a second active ingredient that is at least partially surrounded by both the first capsule and the second capsule. In some embodiments, the second active ingredient can be in the form of a liquid, a solid, or a powder in unit dose form. In some embodiments, the second active ingredient is in the form of a liquid in unit dose form, wherein the liquid is an oil. In some embodiments, the second active ingredient can comprise particles. In some embodiments, the particles may be at least partially encapsulated by the coating material and the particles at least partially encapsulated by the coating material can be spray dried. In some embodiments, at least a portion of the particles at least partially encapsulated by the coating material can have a particle diameter ranging from about 50 micrometers to about 150 micrometers, as measured by a particle analyzer using laser diffraction. In some embodiments, the first active ingredient, the pharmaceutically acceptable salt thereof, the second active ingredient, the pharmaceutically acceptable salt thereof or any combination thereof can be used to treat or prevent a disease or a condition or can be useful for maintaining health. In some embodiments, the first capsule, the second capsule, or both can comprise a capsule coating. In some embodiments, the capsule coating can at least partially control active ingredient release. In some embodiments, the capsule coating can comprise an enteric coating, a time release coating, a pH dependent coating, a delayed release coating, an extended-release coating, or a combination thereof. In some embodiments, the first capsule and the second capsule can be formulated to deliver their contents at different locations in the gastrointestinal system. In some embodiments, the first capsule and the second capsule can be formulated to deliver their contents at about the same location in the gastrointestinal system. In some embodiments, the particles at least partially encapsulated by the coating material can comprise dissolving the particles comprising the active ingredient or the pharmaceutically acceptable salt thereof in a solvent with the coating material. In some embodiments, the coating material can comprise the hydroxypropyl methylcellulose acetate succinate (“HPMCAS”). In some embodiments, the spray drying process can comprise: atomizing liquid droplets comprising the active ingredient or the pharmaceutically acceptable salt thereof and the coating material, drying the droplets to form particles, and recovering the particles. In some embodiments, the spray drying process can comprise one or more active ingredients. In some embodiments, the first active ingredient, the pharmaceutically acceptable salt thereof, the second active ingredient, the pharmaceutically acceptable salt thereof, or any combination thereof can be at least partially embedded in the first capsule, the second capsule or both. In some embodiments, at least a portion of the excipient and the plurality of spray dried particles can be admixed in a substantially homogenous mixture in unit dose form. In some embodiments, at least a portion of the excipient and the second active ingredient, or a pharmaceutically acceptable salt thereof can be admixed in a substantially homogenous mixture in unit dose form. In some embodiments, the pharmaceutical or dietary supplement composition can further comprise adding a capsule band to the first capsule, the second capsule, or both wherein the capsule band at least partially seals the capsule. In some embodiments, the portion of the first capsule not containing the particles can comprise the first active ingredient, the pharmaceutically acceptable salt thereof, the second active ingredient, the pharmaceutically acceptable salt thereof, or any combination thereof can be at least partially filled with an inert gas. In some embodiments, a space can exist between the first capsule and the second capsule, and wherein the space is at least in part filled with an inert gas. In some embodiments, a space can exist between the first capsule and the second capsule, and wherein the space is at least in part filled with an inert gas. In some embodiments, the inert gas can comprise nitrogen, carbon dioxide, helium, or any combination thereof. In some embodiments, the pharmaceutical or dietary supplement composition can comprise a weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 1000:1 (w/w). In some embodiments, the weight to weight ratio can be of a) the excipient and b) the first active ingredient, the pharmaceutically acceptable salt thereof, the second active ingredient, the pharmaceutically acceptable salt thereof, or any combination thereof ranges from about 1:1 (w/w) to about 10:1 (w/w). In some embodiments, a content of the first capsule, the second capsule, or both can comprise less than about 10% water by weight or wherein a total content of all gases in the capsule is less than about 10% water by weight. In some embodiments, the first capsule, the second capsule, or both can comprise a hydroxypropyl methylcellulose (HPMC). In some embodiments, the first capsule size can be: 000, 00, 0, 1, 2, 3, 4, or 5. In some embodiments, the first capsule size can be: 1, 2, 3, 4, or 5. In some embodiments, the second capsule size can be: 000, 00, 0, 1, 2, 3, or 4. In some embodiments, the second capsule size can be: 000, 00, 0, or 1. In some embodiments, the pharmaceutical or dietary supplement composition can be stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC. In some embodiments, in a human clinical trial or human safety trial, the pharmaceutical or dietary supplement composition, when ingested, can operate mechanistically such that in at least a portion of the humans in the clinical trial or the safety trial, at least a portion of the second active ingredient is released earlier in time than the particles comprising the first active ingredient. In some embodiments, the excipient can comprise honey, a glycerin, an oil, a long chain sugar, a polyethylene glycol, a corn syrup, a maltodextrin syrup, a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, the pharmaceutical or dietary supplement composition can comprise an excipient and the excipient can comprise honey. In some embodiments, the excipient can comprise an oil, and the oil can comprise a food oil. In some embodiments, the food oil can comprise an almond oil, an avocado oil, a brazil nut oil, a canola oil, a cashew oil, a chia seed oil, a cocoa butter oil, a coconut oil, a corn oil, a cottonseed oil, a flaxseed/linseed oil, a grape seed oil, a hemp seed oil, a vigna mungo oil, a mustard oil, an olive oil, a bean oil, a palm oil, a peanut oil, a pecan oil, a perilla oil, a rice bran oil, a safflower oil, a sesame oil, a soybean oil, a soybean oil, a walnut oil, a sunflower oil, a cottonseed oil, a vegetable oil or any combination thereof. In some embodiments, the excipient can comprise a carbohydrate, and the carbohydrate can comprise a lactose, a microcrystalline cellulose, a cellulose, a mannitol, a sorbitol, a starch, a starch a glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof. In some embodiments, the first active ingredient can be in the form of a liquid in unit dose form. In some embodiments, the liquid can comprise an oil. In some embodiments, the excipient can comprise a glycerin. In some embodiments, the second active ingredient or the pharmaceutically acceptable salt thereof can comprise a cannabinoid or a pharmaceutically acceptable salt thereof in unit dose form. In some embodiments, the cannabinoid can comprise a tetrahydrocannabinol (THC), a tetrahydrocannabinol Delta-8, a tetrahydrocannabinol Delta-9, a tetrahydrocannabinol Delta-11, a tetrahydrocannabinol Delta-13, a tetrahydrocannabivarin (THCV), a tetrahydrocannabinol THCA, a full spectrum THC, or a pharmaceutically acceptable salt thereof in unit dose form. In some embodiments, the cannabinoid can comprise a cannabichromene (CBC), a cannabichromevarin (CBCV), a cannabidiol (CBD), a cannabidivarin (CBDV), a cannabigerol (CBG), a cannabigerivarin (CBGV), a cannabinol (CBN), a cannabivarin (CBV), a THC Delta-8, or a pharmaceutically acceptable salt thereof in unit dose form. In some embodiments, the cannabichromene (CBC), the cannabichromevarin (CBCV), the cannabidiol (CBD), the cannabidivarin (CBDV), the cannabigerol (CBG), the cannabigerivarin (CBGV), the cannabinol (CBN), the cannabivarin (CBV), the THC Delta-8, or the pharmaceutically acceptable salt thereof in unit dose form can be derived from hemp. In some embodiments, the first active ingredient or the pharmaceutically acceptable salt thereof in unit dose form can be present in an amount ranging from about 1 mg to about 200 mg, or from about 2 mg to about 50 mg. In some embodiments, the second active ingredient or the pharmaceutically acceptable salt thereof in unit dose form can be present in an amount ranging from about 1 mg to about 200 mg, or from about 2 mg to about 50 mg. In some embodiments, the pharmaceutical or dietary supplement composition can further comprise a further active ingredient or a pharmaceutically acceptable salt thereof in unit dose form. In some embodiments, the further active ingredient can comprise a cannabinoid, pharmaceutically acceptable salt thereof, or both in unit dose form. In some embodiments, the further active ingredient can comprise the cannabinoid, wherein the cannabinoid comprises a cannabichromene (CBC), a cannabichromevarin (CBCV), a cannabidiol (CBD), a cannabidivarin (CBDV), a cannabigerol (CBG), a cannabigerivarin (CBGV), a cannabinol (CBN), a cannabivarin (CBV), a THC Delta-8, a tetrahydrocannabinol (THC), a tetrahydrocannabinol Delta-8, a tetrahydrocannabinol Delta-9, a tetrahydrocannabinol Delta-11, a tetrahydrocannabinol Delta-13, a tetrahydrocannabivarin (THCV), a tetrahydrocannabinol THCA, a full spectrum THC, a pharmaceutically acceptable salt of any of these, or any combination thereof in unit dose form. In some embodiments, the pharmaceutical or dietary supplement composition can comprise the salt of the active ingredient. In some embodiments, the salt can comprise an organic salt, an inorganic salt, or any combination thereof. In some embodiments, the salt can comprise an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof. In some embodiments, the excipient can be a pharmaceutically acceptable excipient. In some embodiments, the excipient can be a dietary supplement acceptable excipient. In some embodiments, the pharmaceutical or dietary supplement composition is a pharmaceutical composition. In some embodiments, the pharmaceutical composition can be in a therapeutically effective amount to treat a disease or condition. In some embodiments, the pharmaceutical or dietary supplement composition is a dietary supplement composition. In some embodiments, the dietary supplement composition can be in effective amount to maintain health of a subject.

Also disclosed herein are kits that can comprise pharmaceutical or dietary supplement compositions described herein. In some embodiments, the kit can be contained at least in part in a packaging.

Also disclosed herein are methods of making kits that can comprise at least partially packaging the pharmaceutical or dietary supplement compositions described herein into a packaging.

Also described herein are methods of treating or preventing a disease or condition in a subject in need thereof. In some embodiments the methods of treating or preventing a disease or condition in a subject in need thereof can comprise treating or preventing the disease or condition by administering a therapeutically effective amount of a pharmaceutical composition described herein. In some embodiments, administering can be conducted one, two, three, or four times per day. In some embodiments, the disease or condition can be selected from the group consisting of: cancer, breast cancer, melanoma, anxiety, pruritus (itching), cognitive function, Alzheimer's disease, amyotrophic lateral sclerosis, Huntington's disease, Parkinson's disease, a chronic pain, pain management, multiple sclerosis, a side effect of chemotherapy, HIV, AIDS, a neurodegenerative disorder, a behavior disorder, Tourette syndrome, cervical dystonia, sleep aide, appetite stimulant, a seizure, an epilepsy, nausea associated with chemotherapy, anorexia, spinal cord injury, sleep disorders, glaucoma, schizophrenia, epilepsy, asthma, posttraumatic stress disorder, cachexia, irritable bowel syndrome, a dependency withdrawal, psychiatric symptoms, autoimmune diseases, inflammation, sleep apnea, a headache, a migraine, opioid addiction, and any combination thereof. In some embodiments, the pharmaceutical composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically. In some embodiments, the amount of the active ingredient or the pharmaceutically acceptable salt thereof in the unit dose can range from about 1 mg to about 200 mg, or from about 2 mg to about 50 mg. In some embodiments, a second therapeutic or a pharmaceutically acceptable salt thereof can be administered. In some embodiments, the second therapeutic or a pharmaceutically acceptable salt thereof can be administered concurrently. In some embodiments, the second therapeutic or the pharmaceutically acceptable salt thereof can be comprised in the pharmaceutical formulation. In some embodiments, the second therapeutic or the pharmaceutically acceptable salt thereof may not be comprised in the pharmaceutical formulation. In some embodiments, the second therapeutic or the pharmaceutically acceptable salt thereof can be administered consecutively. In some embodiments, subject can be diagnosed with the disease or condition. In some embodiments, diagnosing can comprise employing an in vitro diagnostic. In some embodiments, the in vitro diagnostic can be a companion diagnostic. In some embodiments, the administering can be oral administration, rectal administration, or any combination thereof. In some embodiments, subject can be a non-human animal. In some embodiments, the non-human animal can be a cat, a dog, a horse, a rodent, a cow, a pig, a bird, a goat, a sheep, or a reptile. In some embodiments, subject can be a human. In some embodiments, subject can be born as a man. In some embodiments, subject can be born as a woman. In some embodiments, the subject can be over 18 years of age. In some embodiments, the subject can be under 18 years of age. In some embodiments, the second therapeutic or the pharmaceutically acceptable salt thereof can be administered orally, intranasally, intraocularly, anally, by injection, intravenously, intramuscularly, subcutaneously, intraperitoneally, trans dermally, or any combination thereof.

Also disclosed herein are methods of making the pharmaceutical or dietary supplement compositions described herein. In some embodiments, the method can comprise formulating a capsule-in-capsule formulation in unit dose form wherein the formulation can comprise: a first active ingredient or a pharmaceutically acceptable salt thereof that can comprise a plurality of spray dried particles, each particle of the plurality of spray dried particles comprising a cannabinoid or a pharmaceutically acceptable salt thereof, substantially encapsulated in a coating material. In some embodiments, the coating material can comprise a hydroxypropyl methylcellulose (HPMC), a hydroxypropyl methylcellulose acetate succinate (HPMCAS), a maltodextrin, a povidone, a copovidone or any combination thereof. In some embodiments, the plurality of spray dried particles can be at least partially surrounded by a first capsule, a second capsule, or both; and wherein the first capsule is surrounded by the second capsule. In some embodiments, the pharmaceutical or dietary supplement composition further can comprise a second active ingredient, or a pharmaceutically acceptable salt thereof in unit dose form. In some embodiments, the pharmaceutical or dietary supplement composition further can comprise an excipient. In some embodiments, the second active ingredient can be at least partially surrounded by the first capsule, the second capsule or both. In some embodiments, the second active ingredient in unit dose form is in the form of a liquid, a solid, or a powder. In some embodiments, the second active ingredient can comprise particles. In some embodiments, the particles can be at least partially encapsulated by a coating material and wherein the particles at least partially encapsulated by the coating material are spray dried. In some embodiments, at least a portion of the particles at least partially encapsulated by the coating material can have a particle diameter ranging from about 50 micrometers to about 150 micrometers, as measured by a particle analyzer using laser diffraction. In some embodiments, the first capsule, the second capsule, or both can comprise a capsule coating. In some embodiments, the capsule coating can at least partially controls active ingredient release. In some embodiments, the capsule coating can comprise an enteric coating, a time release coating, a pH dependent coating, a delayed release coating, an extended release coating, or a combination thereof. In some embodiments, the at least partially encapsulated by the coating material can comprise dissolving the particles comprising the active ingredient or the pharmaceutically acceptable salt thereof in a solvent with the coating material. In some embodiments, the capsule coating comprises an enteric coating. In some embodiments, the method can further comprise adding a capsule band to the first capsule, the second capsule, or both, wherein the capsule band seals the capsule. In some embodiments, the portion of the first capsule not containing the particles can comprise the first active ingredient, the pharmaceutically acceptable salt thereof, the second active ingredient, the pharmaceutically acceptable salt thereof, or any combination thereof is at least partially filled with an inert gas. In some embodiments, a space can exist between the first capsule and the second capsule, and wherein the space is at least in part filled with an inert gas. In some embodiments, the inert gas can comprise nitrogen, carbon dioxide, helium, or any combination thereof. In some embodiments, the first capsule, the second capsule, or both can be containers that can comprise the pharmaceutical or dietary supplement formulation.

### BRIEF DESCRIPTION OF THE DRAWINGS

FIG. 1 shows a spray drying manufacturing system comprising a closed spray drying chamber which receives a solution comprising an active ingredient microencapsulated in a polymer in a suitable solvent. The system generates dried microencapsulated particles from the solution comprising the microencapsulated active ingredient.

### DETAILED DESCRIPTION

### Overview

Delivering pharmaceutical or dietary supplement compositions through oral ingestion of capsules or tablets can be an effective way to administer a drug such as a cannabinoid. The capsule-in-capsule technology as described herein can be used to release an active compound or salt thereof to different (or the same) locations of the gastrointestinal tract. By spray drying the compositions the particle sizes can be controlled to increase dosing precision.

Provided herein are pharmaceutical or dietary supplement compositions, kits comprising pharmaceutical or dietary supplement compositions, methods of treating and preventing disease, and methods of making compositions and kits described herein. Pharmaceutical drugs and dietary supplements described herein can be produced employing various methods to synthesize, manipulate, and administer particles.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be a pet or livestock. The subject may be diagnosed or suspected of being at high risk for a disease. In some cases, the subject may not be necessarily diagnosed or suspected of being at high risk for the disease.

The term “substantially” or “essentially” can refer to a qualitative condition that exhibits an entire or nearly total range or degree of a feature or characteristic of interest. In some cases, substantially encapsulated can refer to near complete encapsulation of a substance or compound. For example, substantially encapsulated can comprise a particle that is at least about: 70%, 75%, 80%, 85%, 90%, 95%, 99%, or 100% encapsulated. In some cases, substantially can refer to at least about: 70%, 75%, 80%, 85%, 90%, 95%, 99%, or 100% of the total range or degree of a feature or characteristic of interest.

The term “at least partially” can refer to a qualitative condition that exhibits a partial range or degree of a feature or characteristic of interest. In some cases, at least partially encapsulated can refer to a partial encapsulation of a substance or compound. For example, at least partially encapsulated can comprise a particle that is at least about: 5%, 10%, 20%, 30%, 40%, 50%, 60%, 70%, 75%, 80%, 85%, 90%, 95%, 99%, or 100% encapsulated.

The term “in vivo” can be used to describe an event that takes place in a subject's body.

The term “ex vivo” can be used to describe an event that takes place outside of a subject's body. An “ex vivo” assay may not be performed on a subject. Rather, it can be performed upon a sample separate from a subject. An example of an “ex vivo” assay performed on a sample can be an “in vitro” assay.

The term “in vitro” can be used to describe an event that takes place contained in a container for holding laboratory reagent such that it can be separated from the living biological source organism from which the material may be obtained. In vitro assays can encompass cell-based assays in which cells alive or dead are employed. In vitro assays can also encompass a cell-free assay in which no intact cells are employed.

As used herein, the term “about” a number can refer to that number plus or minus 10% of that number. The term “about” a range can refer to that range minus 10% of its lowest value and plus 10% of its greatest value. In some cases, the term “about” a number can refer to that number plus or minus 20% of that number. The term “about” a range can refer to that range minus 20% of its lowest value and plus 20% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “unit dose” or “dosage form” can be used interchangeably and can be meant to refer to pharmaceutical drug products or dietary supplements in the form in which they are marketed for use, with a specific mixture of active ingredients and inactive components or excipients, in a particular configuration, and apportioned into a particular dose to be delivered. The term “unit dose” can also sometimes encompass non-reusable packaging, although the FDA distinguishes between unit dose “packaging” or “dispensing”. More than one unit dose can refer to distinct pharmaceutical drug products or dietary supplement products packaged together, or to a single pharmaceutical drug product or dietary supplement product containing multiple active compounds and/or doses. The term “unit dose” can also sometimes refer to the particles comprising a pharmaceutical composition, a dietary composition and to any mixtures involved. Types of unit doses may vary with the route of administration for drug or supplement delivery, and the substance(s) being delivered. A solid unit dose can be the solid form of a dose of a chemical compound used as a pharmaceutically acceptable drug or medication intended for administration or consumption. A solid unit dose can be the solid form of a dose of a chemical compound used as an acceptable dietary supplement intended for administration or consumption.

As used herein, a “dose” can refer to a measured quantity of a therapeutic agent to be taken at one time.

As used herein, “pharmaceutically acceptable salt” can refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, “laser diffraction” can refer to a method for particle size analysis, which consists of scattering laser light off an assembly of particles, and collecting the scattered light using a spatial array of detectors. The signal from the detectors can be a pattern of scattered/diffracted light vs. angle. This pattern can result from many particles being illuminated by the laser light source at the same time, where all of their individual scattered/diffracted light rays mix together at each detector element.

As used herein, “particle size analyzer” can refer to an instrument for particle size analysis, particle size measurement, or simply particle sizing.

As used herein, “particle size analysis” can refer to the collective name of the technical procedures, or laboratory techniques which determines the size range, and/or the average (mean), median or mode size of the particles in a powder or liquid sample.

As used herein, “time to peak plasma concentration” can refer to the time required for a drug to reach peak concentration in plasma. Peak concentration in plasma can be usually defined as the plasma concentration that a drug achieves in a specified compartment or test area of the body after the drug has been administered and before the administration of a second dose.

As used herein, “HPLC” can refer to high-performance liquid chromatography (formerly referred to as high-pressure liquid chromatography), which is a technique in analytical chemistry used to separate, identify, and quantify each component in a mixture. HPLC can be a common technique used in pharmaceutical development, as it can be a method to ensure product purity.

As used herein, the terms “effective amount” or “therapeutically effective amount” of a drug used to treat a disease can be an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose.

As used herein, the term “substantially” can refer to a degree of deviation that is sufficiently small so as to not measurably detract from the identified property or circumstance. In some cases, the exact degree of deviation allowable may in some cases depend on the specific context.

The section headings used herein are for organizational purposes only and are not to be construed as limiting the subject matter described.

### Compositions

In some embodiments, disclosed herein are devices, systems and methods for producing, packaging, and delivering stable pharmaceutical and dietary supplement compositions to a body via capsules. In some embodiments, a composition can be a pharmaceutical composition. A pharmaceutical composition can be administered to a human or to an animal. In some embodiments, a composition can be a dietary supplement composition. In some embodiments, a composition can be a composition for the non-human animal. In some embodiments, a composition can be in unit dose form. In some embodiments, a dietary supplement composition can be administered to a human. In some embodiments, a dietary supplement composition can comprise a supplement for use in an animal (e.g. a pet). In some embodiments, a dietary supplement composition can be administered to an animal. In some embodiments, a composition can be spray dried. In those embodiments, the addition of an excipient carrier product to the active powders described herein prior to encapsulation can improve stability and effective solubility.

In some embodiments, a composition can be a pharmaceutical or dietary supplement composition, in unit dose form. In some embodiments, a composition can be a composition for a non-human animal. In some embodiments a pharmaceutical or dietary supplement composition can comprise i) particles comprising a first active ingredient, or a pharmaceutically acceptable salt thereof wherein the particles are at least partially encapsulated by a coating material and wherein the particles at least partially encapsulated by the coating material are spray dried. In some cases, the particles from i) can be at least partially surrounded by a first capsule, a second capsule, or both. In some cases, the first capsule can be surrounded by a second capsule. In some cases, the composition can be in a capsule-in-capsule. In some cases, the composition can further comprise a second active ingredient, or a pharmaceutically acceptable salt thereof in unit dose form, an excipient, or both. In some cases, a capsule can comprise a capsule coating. In some cases, a capsule coating can at least partially control active ingredient release.

In some embodiments the compositions can comprise one or more of: an active ingredient or salts, excipients, and inactive ingredients. In some cases, a composition can comprise a first active ingredient. In some cases, a composition can comprise a second active ingredient. In some cases, an active ingredient or salt thereof can comprise a particle. In some cases, an active ingredient or salt thereof can be in the form of a powder, a liquid, or a solid. In some cases, an active ingredient or salt thereof can be an oil. In some cases, a composition can comprise particles. In some cases, a composition can be in the form of a particle. In some cases, the particles can be encapsulated. In some cases, particles can comprise an excipient (e.g. a pharmaceutically or dietary supplement acceptable excipient) or an active ingredient. In some cases, a composition can comprise a pharmaceutical composition, a dietary supplement composition, or a composition for a non-human animal. In some cases, an active ingredient or salt thereof can treat or prevent a disease or condition. In some cases, an active ingredient or salt thereof can be useful for maintaining health. In some instances, a composition can comprise an excipient. In some cases, an excipient can be a pharmaceutically acceptable excipient. In some cases, an excipient can be a dietary supplement acceptable excipient. In some instances, a composition can comprise particles comprising an active ingredient, or a pharmaceutically acceptable salt thereof at least partially encapsulated by a coating material and wherein the particles at least partially encapsulated by the coating material are spray dried. In some embodiments, the first active ingredient, the second active ingredient or both can be at least partially surrounded by a first capsule, a second capsule or both. In some cases, the first active ingredient, the salt thereof, the second active ingredient, the salt thereof, or any combination thereof is at least partially embedded in the first capsule, the second capsule or both. For example, an active ingredient can be embedded in a hydroxypropyl methylcellulose capsule.

As used herein, “coating material” can refer to a material added via a pharmaceutical coating process by which an essentially dry, outer layer of coating material can be applied to the surface of a dosage form. In some cases, a coating material can be added to a dosage form prior to or after spray drying. In some cases, coating dosage forms may be used to improve stability (light protection, moisture and gas barrier), facilitate administration, or modify the drug release behavior from the dosage form. Coating materials may include film coating formulations, which usually contain a polymer, a plasticizer, a colorant, opacifier, a solvent, and a vehicle.

In some embodiments, a composition can comprise a mixture of particles described herein. In some embodiments, the particles can be mixed in a substantially homogenous mixture. In some embodiments, an active ingredient and an excipient can be mixed in a substantially homogenous mixture. In some embodiments, an excipient can be mixed in the particles described herein. In some instances, at least a portion of the particles comprising an active ingredient or a pharmaceutically acceptable salt thereof at least partially encapsulated by a coating material can have a particle diameter ranging from about 30.0 micrometers to about 200 micrometers or about 50.0 micrometers to about 150.0 micrometers, as measured by a particle size analyzer using laser diffraction.

In some embodiments, in a human clinical trial, a human safety trial, a veterinary clinical trial, or an animal safety trial when ingested, a composition described herein operates mechanistically such that in at least a portion of the humans or animals in the trial, at least a portion of the particles comprising the second active ingredient can be released earlier in time than the particles comprising the first active ingredient. In some embodiments, the particles comprising the first active ingredient can be comprised in a first capsule. In some embodiments, the particles comprising the second active ingredient can be comprised in a second capsule. In some embodiments, a first capsule can be comprised in a second capsule. In some embodiments, the particles comprising the second active ingredient or the salt thereof can be released from about 1 min to about 20 min, about 10 min to about 60 min, about 20 min to about 120 min, about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 5 hours to about 15 hours, about 5 hours to about 20 hours, about 5 hours to about 30 hours, about 5 hours to about 35 hours, about 10 hours to about 20 hours, about 10 hours to about 15 hours, about 10 hours to about 20 hours, about 10 hours to about 30 hours, about 10 hours to about 35 hours, about 20 hours to about 30 hours, or about 20 hours to about 35 hours earlier than the particles comprising the first active ingredient. In some embodiments, a composition comprised in the second capsule can be released from about 1 min to about 20 min, about 10 min to about 60 min, about 20 min to about 120 min, about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 5 hours to about 15 hours, about 5 hours to about 20 hours, about 5 hours to about 30 hours, about 5 hours to about 35 hours, about 10 hours to about 20 hours, about 10 hours to about 15 hours, about 10 hours to about 20 hours, about 10 hours to about 30 hours, about 10 hours to about 35 hours, about 20 hours to about 30 hours, or about 20 hours to about 35 hours earlier than a composition comprised in the first capsule.

In some embodiments, the weigh to weight ratio of: a) an excipient and b) an active ingredient or a pharmaceutically acceptable salt thereof ranges from about 1:1 to about 10000:1. In some embodiments, the weight to weight ratio of: a) an excipient and b) an active ingredient or a pharmaceutically acceptable salt thereof can range from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some embodiments, the weight to weight ratio of: a) an excipient and b) an active ingredient or a pharmaceutically acceptable salt thereof can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some embodiments, the weight to weight ratio of: a) an excipient and b) an active ingredient or a pharmaceutically acceptable salt thereof can range from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10. In some cases, the active ingredient can be the first active ingredient, the second active ingredient or both. In some cases, the active ingredient can comprise more than two active ingredients.

### Active Ingredients and Pharmaceutically Acceptable Salts

In some embodiments, an active ingredient can comprise an active pharmaceutical ingredient or an active dietary supplement ingredient. In some embodiments, an active ingredient can comprise an active pharmaceutical ingredient. In some cases, an active pharmaceutical ingredient can be any substance or mixture of substances intended to be used in the manufacture of a drug (medicinal) product and that, when used in the production of a drug, becomes an active ingredient of the drug product. Such substances can be intended to furnish pharmacological activity or other direct effect in the diagnosis, cure, mitigation, treatment, or prevention of disease or to affect the structure or function of the body. In some cases, an active ingredient can comprise a pharmaceutical compound. In some cases, a pharmaceutical compound can comprise an active ingredient.

In some embodiments, an active ingredient can comprise an active dietary supplement ingredient. In some cases, an active dietary supplement ingredient can be any substance or mixture of substances intended to be used in the manufacture of a dietary supplement product and that, when used in the production of a dietary supplement, becomes an active ingredient of the dietary supplement. Such substances can be intended to supplement the diet of a subject, boost energy, or maintain the health of a subject.

In some embodiments, an active ingredient or salt thereof can be formulated as oil emulsion. In some embodiments, an active ingredient or salt thereof can be formulated as an oil, a liquid, or a gel. In some embodiments, an active ingredient or salt thereof can be encapsulated by a coating material and can be spray dried. In some cases, an active ingredient can include more than one compound. In some cases, different active ingredients can be comprised in a capsule. In some cases, different active ingredients can be independently separated into the inner capsule, the outer capsule, or both of a capsule-in-capsule capsule. In some embodiments, an active ingredient or salt thereof can comprise a cannabinoid or a salt thereof. The term “cannabinoid” can refer to a chemical compound that shows direct or indirect activity at a cannabinoid receptor. In some instances, a cannabinoid can comprise a phytocannabinoid. In some instances, a cannabinoid can comprise a endocannabinoid. In some instances, an endocannabinoid can comprise anandamide (arachidonoyl ethanolamide) or 2-arachidonoyl glycerol (2-AG). In some cases, a cannabinoid can be a full spectrum cannabinoid. In some cases, a cannabinoid can be a broad-spectrum cannabinoid. Examples of cannabinoids include, but are not limited to, a Tetrahydrocannabinol, commonly referred to as “THC”, an isolate THC, or a full spectrum THC, a cannabidiol (CBD), a cannabinol (CBN), a cannabigerol (CBG), a cannabichromene (CBC), a cannabicyclol (CBL), a cannabivarin (CBV), a cannabidivarin (CBDV), a cannabichromevarin (CBCV), a cannabigerovarin (CBGV), a cannabigerol monomethyl ether (CBGM), a cannabidiolic acid (CBDA) and salt thereof. Other examples can include Tetrahydrocannabinol Delta-8, Tetrahydrocannabinol Delta-9, Tetrahydrocannabinol Delta-11, Tetrahydrocannabinol Delta-13, a Tetrahydrocannabivarin (THCV), a Tetrahydrocannabinol (THCA) and salt thereof. In some instances, a cannabinoid or a salt thereof can be derived from hemp. For example, the CBC, CBCV, CBD, CBDV, CBG, CBGV, CBN, CBV, THC Delta-8, or pharmaceutically acceptable salts thereof can be derived from hemp. In some instances, a cannabinoid or a salt thereof can be derived from Cannabis. In some instances, a tetrahydrocannabinol or a salt thereof can be derived from hemp. In some instances, tetrahydrocannabinol Delta-8 or a salt thereof can be derived from hemp. In some instances, tetrahydrocannabinol Delta-8 or a salt thereof can be derived from Cannabis. In some cases, a cannabinoid can be a synthetic cannabinoid or a salt thereof. In some cases, a cannabinoid can include a derivative of a cannabinoid or a salt thereof. In some instances, a cannabinoid can comprise an isomer of a cannabinoid. A derivative of a compound disclosed herein, can refer to a chemical substance related structurally to a compound disclosed herein. A derivative can be made from a structurally related parent compound in one or more steps. In some cases, the general physical and chemical properties of a derivative can be similar to a parent compound.

Tetrahydrocannabinol (THC) is one of at least 113 cannabinoids identified in Cannabis. THC can be a psychoactive constituent of cannabis. The term THC can also refer to cannabinoid isomers, for example (−)-trans-Δ⁹-tetrahydrocannabinol. THC can be a lipid found in Cannabis. In some cases, a cannabinoid can comprise a cannabinoid from Table 1 and/or Table 2.

Examples of cannabinoids are shown in Table 1 and in Table 2.

TABLE 1
Cannabinoid Examples

| Cannabinoid Name | Formula | Molar Mass | | Structure |
|---|---|---|---|---|
| Tetrahydrocannabinol, THC | C₂₁H₃₀O₂ | 314.45 | g/mol | 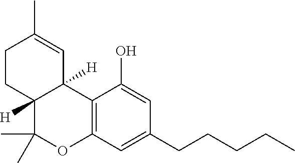 [SMILES: CCCCCc1cc(O)c2c(c1)OC(C)(C)[C@@H]1CCC(C)=C[C@@H]21] |
| Tetrahydrocannabivarin, THCV | C₁₉H₂₆O₂ | 286.415 | g/mol | 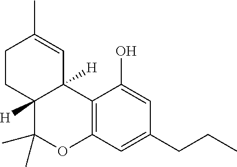 [SMILES: CCCc1cc(O)c2c(c1)OC(C)(C)[C@@H]1CCC(C)=C[C@@H]21] |
| Delta-8* | C₂₁H₃₀O₂ | 314.5 | g/mol | 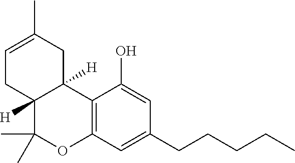 [SMILES: CCCCCc1cc(O)c2c(c1)OC(C)(C)[C@@H]1CC=C(C)C[C@@H]21] |
| Delta-9* | C₂₁H₃₀O₂ | 314.45 | g/mol | 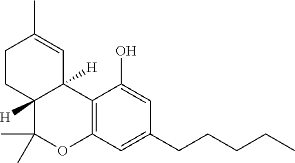 [SMILES: CCCCCc1cc(O)c2c(c1)OC(C)(C)[C@@H]1CCC(C)=C[C@@H]21] |
| Tetrahydrocannabinolic acid, THCA | C₂₂H₃₀O₄ | 358.478 | g/mol | 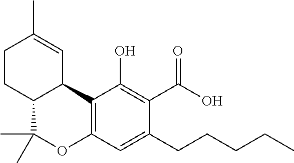 [SMILES: CCCCCc1cc2c(c(O)c1C(=O)O)[C@@H]1C=C(C)CC[C@H]1C(C)(C)O2] |
| Cannabidiol, CBD | C₂₁H₃₀O₂ | 314.47 | g/mol | 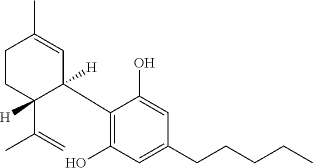 [SMILES: C=C(C)[C@@H]1CCC(C)=C[C@H]1c1c(O)cc(CCCCC)cc1O] |
| Cannabigerol, CBG | C₂₁H₃₂O₂ | 316.48 | g/mol | 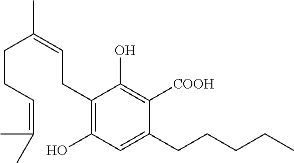 [SMILES: CCCCCc1cc(O)c(C/C=C(/C)CCC=C(C)C)c(O)c1C(=O)O] |
| Cannabichromene, CBC | | | | |
| 11-Hydroxy-THC | C₂₁H₃₀O₃ | 330.468 | g/mol | 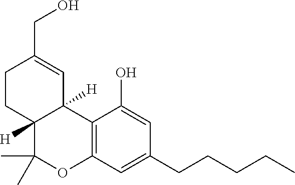 [SMILES: CCCCCc1cc(O)c2c(c1)OC(C)(C)[C@@H]1CCC(CO)=C[C@@H]21] |
| Cannabinol, CBN | C₂₁H₂₆O₂ | 310.4319 | g/mol | 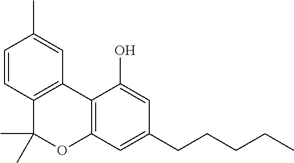 [SMILES: CCCCCc1cc(O)c2c(c1)OC(C)(C)c1ccc(C)cc1-2] |

*Delta-8 and Delta-9 have a subtle difference in the double bond in their molecular structure.

TABLE 2
List of exemplary cannabinoids

| | Abbrevi- |
|---|---|
| Cannabinoid | ation |
| Cannabigerovarin | CBGV |
| Cannabigerovarinic acid | CBGVA |
| Cannabigerol | CBG |
| Cannabigerolic acid | CBGA |
| O-Methlycannabigerol | |
| Cannabigerolic acid methylether | |
| Cannabinerolic acid | |
| Cannabinerol | |
| Carmagerol | |
| rac-6′-Epoxycannabigerol | |
| rac-6′-Epoxycannabigerolic acid | |
| rac-6′-Epoxycannabinerol | |
| rac-6′-Epoxycannabinerolic acid | |
| γ-Eudesmyl cannabigerolate | |
| γ-Cadinyl cannabigerolate | |
| Sesquicannabigerol | |
| Deprenyl O-methyl cannabigerolic acid | |
| (Amorfrutin 2) | |
| 5-Acetyl-4-hydroxycannabigerol | |
| Acetylcannabigeroquinol | |
| Cannabigeroquinone | |
| Abnormal cannabigerol | |
| Acetyl abnormal hydrocannabigeroquinol | |
| Abnormal cannabigeroquinol | |
| 2′-Hydroxy-1′,2′-dihydrocannabichromene | |
| (Cyclo-CBG) | |
| Cannabiorcichromene | |
| Cannabiorcichromenic acid | |
| Chlorcannabiorcichromenic acid | |
| Cannabivarichromene | |
| Cannabichromevarinic acid | |
| Cannabichromene | CBC |
| Cannabichromenic acid | |
| 4-Acetoxycannabichromene | |
| Anthopogochromenic acid | |
| Confluentin | |
| Daurichromenic acid | |
| 8′-Hydroxyisocannabichromene | |
| 4-Acetoxycannabichromene | |
| Cannabidiorcol | |
| Cannabidivarin | CBDV |
| Cannabidivarinic acid | |
| nor-Cannabidiol | |
| Cannabidiol | CBD |
| O-Methlycannabidiol | |
| O-Propylcannabidiol | |
| O-Pentylcannabidiol | |
| Cannabidiolic acid | CBDA |
| CBDA-THC ester | |
| Ferruginene C | |
| Cannabioxepane | |
| Cannabinodivarin | |
| Cannabinodiol | |
| Cannabifuran | |
| Dehydrocannabifuran | |
| trans-Δ-8-Tetrahydrocannabinol | |
| trans-Δ-8-Tetrahydrocannabinolic acid | |
| 10α-Hydroxy trans-Δ-8-tetrahydrocannabinol | |
| 10β-Hydroxy trans-Δ-8-tetrahydrocannabinol | |
| 11-Acetoxy-Δ-8-tetrahydrocannabinoic acid | |
| 10-Hydroxy-9-oxo-Δ-8-tetrahydrocannabinol | |
| Δ-9-trans-Tetrahydrocannabiorcol | |
| Δ-9-trans-Tetrahydrocannabiorcolic acid | |
| Δ-9-trans-Tetrahydrocannabivarin | |
| Δ-9-trans-Tetrahydrocannabivarinic acid | |
| Δ-9-trans-nor-Tetrahydrocannabinol | |
| Δ-9-trans-nor-Tetrahydrocannabinolic acid | |
| Δ-9-trans-Tetrahydrocannabinol | |
| Δ-9-trans-Tetrahydrocannabinolic acid A | |
| Δ-9-trans-Tetrahydrocannabinolic acid B | |
| 8α-Hydroxy-Δ-9-trans-tetrahydrocannabinol | |
| 8β-Hydroxy-Δ-9-trans-tetrahydrocannabinol | |
| 8-Oxo-Δ-9-trans-tetrahydrocannabinol | |
| O-Propyl-Δ-9-trans-tetrahydrocannabinol | |
| O-Pentyl-Δ-9-trans-tetrahydrocannabinol | |
| 2-Formyl-Δ-9-trans-tetrahydrocannabinol | |
| β-Fenchyl Δ-9-trans-Tetrahydrocannabinolate | |
| α-Fenchyl Δ-9-trans-Tetrahydrocannabinolate | |
| Bornyl Δ-9-trans-Tetrahydrocannabinolate | |
| epi-Bornyl Δ-9-trans-Tetrahydrocannabinolate | |
| α-Terpinyl Δ-9-trans-Tetrahydrocannabinolate | |
| 4-Terpinyl Δ-9-trans-Tetrahydrocannabinolate | |
| γ-Eudesmyl Δ-9-trans-Tetrahydrocannabinolate | |
| α-Cadinyl Δ-9-trans-Tetrahydrocannabinolate | |
| Hexahydrocannabinol | |
| Hydroxy Δ-9,11-hexahydrocannabinol | |
| Methylen-bis Δ-9-trans-Tetrahydrocannabinol | |
| (Cannabisol) | |
| Tetrahydrocannabinol epoxide | |
| Δ-9-trans-Tetrahydrocannabinol glycol | |
| (cannabiripsol) | |
| 6a,7,10a-Trihydroxy-Δ-9-tetrahydrocannabinol | |
| Δ-9-cis-Tetrahydrocannabivarin | |
| Δ-9-cis-Tetrahydrocannabinol | |
| Cannabicitran (citrilidene-cannabis) | CBT |
| Cannabiorcicitran | |
| Bis-nor cannabitriol | |
| Bis-nor-Cannabitriol isomer | |
| 10-O-Ethyl bis-nor cannabitriol | |
| Isocannabitriol | |
| Cannabitriol | |
| Cannabitriol isomer | |
| 10-O-Ethyl cannabitriol isomer | |
| 10-Oxo-Δ6a(10a)-tetrahydrocannabinol | |
| 9,10-Anhydrocannabitirol | |
| Cannabiglendol | |
| 7,8-Dehydro-10-O-ethylcannabitriol | |
| Δ-7-cis-Isotetrahydrocannabivarin | |
| Δ-7-trans-Isotetrahydrocannabivarin | |
| Δ-7-trans-isotetrahydrocannabinol | |
| Bis-nor-cannabielsoin | |
| Bis-nor-Cannabielsoic acid B | |
| Cannabielsoin | CBE |
| Cannabielsoic acid A | |
| Cannabielsoic acid B | |
| Ferruginene A | |
| Ferruginene B | |
| Cannabiorcicyclol | |
| Cannabiorcicyclolic acid | |
| Cannabicyclovarin | |
| Cannabicyclol | CBL |
| Cannabicyclolic acid | |
| Anthopogocyclolic acid | |
| Rhododaurichromanic acid A | |
| Cannabiorcol | |
| nor-Cannabivarin | |
| Cannabivarin | CBV |
| nor-Cannabinol | |
| Cannabinol | CBN |
| Cannabinolic acid | |
| O-Methylcannabinol | |
| O-Propylcannabinol | |
| O-Penthylcannabinol | |
| 7-Hydroxcannabinol | |
| 8-Hydroxycannabinol | |
| 8-Hydroxcannabinolic acid | |
| 7,8-Dihydrocannabinol | |
| 4-Terpenyl cannabinolate | |
| Cannabicoumaronone | |
| Cannabicourmarononic acid | |
| Bisnor-Cannabichromanone | |
| Cannabichromanone | |
| (6aR)-Cannabichromanone B | |
| (6aR)-Cannabichromanone C | |
| Cannabichromanone | |
| Cannabimovone | |
| Anhydrocannabimovone | |
| Demethyldecarboxyamorfrutin A | |
| Tetrahydrocannabiphorol | THCP |
| Cannabidiphorol | CBDP |
| Cannabimovone | CBM |

In some embodiments, an active ingredient or a salt thereof can comprise a THC, a CBD, a THCV, THC Delta-8, CBG, CBN, a pharmaceutically acceptable salt of any of these, or any combination thereof. In some embodiments, an active ingredient can comprise THC or a pharmaceutically acceptable salt thereof. In some embodiments, an active ingredient can comprise CBD or a pharmaceutically acceptable salt thereof. In some embodiments, an active ingredient can comprise THC, and CBD or pharmaceutically acceptable salts thereof. In some embodiments, an active ingredient can comprise THCV, and CBD or pharmaceutically acceptable salts thereof. In some embodiments, an active ingredient can comprise THC Delta-8, and CBD or pharmaceutically acceptable salts thereof. In some embodiments, an active ingredient can comprise CBG, and CBD or pharmaceutically acceptable salts thereof. In some embodiments, an active ingredient can comprise THC Delta-8, and CBG or pharmaceutically acceptable salts thereof. In some embodiments, an active ingredient can comprise CBN, and CBD or pharmaceutically acceptable salts thereof.

Tetrahydrocannabinol, commonly referred to as “THC”, Isolate THC, or Full Spectrum THC, can include Tetrahydrocannabinol Delta-7, Tetrahydrocannabinol Delta-8, Tetrahydrocannabinol Delta-9, Tetrahydrocannabinol Delta-11, Tetrahydrocannabinol Delta-10, Tetrahydrocannabinol Delta-13, Tetrahydrocannabivarin (THCV) and Tetrahydrocannabinolic acid (THCA). In some instances, THC can comprise trans-THC, cis-THC or both. In some cases, THC can exist as a stereoisomer, such as, (+)-trans-THC; (−)-trans-THC; (+)-cis-THC and (−)-cis-THC. In some cases, cis-TCH can comprise, (+)-cis-THC, (−)-cis-THC, or both. In some cases, trans-THC can comprise (+)-trans-THC, (−)-trans-THC, or both. In some cases, a composition can comprise a ratio (weight to weight) of trans-THC to cis-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-THC to trans-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, CBD can comprise trans-CBD. In some cases, trans-CBD can comprise (+)-trans-CBD, (−)-trans-CBD, or both. In some instances, CBD can comprise an enantiomer, or a diastereomer. In some instances, CBD can comprise a racemate. In some instances, CBD can comprise trans-CBD, cis-CBD or both. In some cases, CBD can comprise (1R,6R)-CBD, (1R,6S)-CBD, (1S,6R)-CBD, (1S,6S)-CBD, or a combination thereof. In some cases, a composition can comprise a ratio (weight to weight) of trans-CBD to cis-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-CBD to trans-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, CBD can be a powder, a liquid, an oil, an emulsion, an aerosol, a solid or a combination thereof. In some cases, CBD can be at least partially water soluble. In some instances, a cannabinoid can be a racemate. In some instances, a cannabinoid can comprise an isomer. In some instances, a cannabinoid can comprise an enantiomer, or a diastereomer. In some cases, a composition can comprise a ratio (weight to weight) of THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, CBD can be mixed in a composition with Δ8-THC, Δ9-THC, Δ10-THC or a combination thereof. In some cases, a composition can comprise a ratio (weight to weight) of Δ8-THC, Δ9-THC, or Δ10-THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to Δ8-THC, Δ9-THC, or Δ10-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000.

In some embodiments, an active ingredient or salt thereof can be formulated as a powder. For example, a microencapsulated THC oil disclosed herein can be formulated as a powder using the methods described herein.

In some embodiments, the active pharmaceutical ingredients can comprise phosphodiesterase inhibitors or pharmaceutically acceptable salts thereof. In some embodiments, the phosphodiesterase inhibitors can be phosphodiesterase type 5 inhibitors (PDES inhibitors). In some embodiments, the phosphodiesterase type 5 inhibitors can include Sildenafil Citrate (Viagra), Tadalafil (Cialis) Avanafil (Stendra), and Vardenafil Hydrochloride (Levitra). In some cases, a phosphodiesterase inhibitor can comprise a selective phosphodiesterase inhibitor, a nonselective phosphodiesterase inhibitor, a PDE-I selective inhibitor, a PDE-II selective inhibitor (e.g. EHNA (erythro-9-(2-hydroxy-3-nonyl)adenine)), a PDE-III selective inhibitor, a PDE-IV selective inhibitor, a PDE-V selective inhibitor, a PDE-VI selective inhibitor, a PDE-VII selective inhibitor, a PDE-IX selective inhibitor, a PDE-X selective inhibitor, a PDE-XI selective inhibitor, a pharmaceutically acceptable salt of any of these, or any combination thereof. In some cases, an active pharmaceutical ingredient can comprise oxindole, inamrinone, anagrelide, cilostazol, mesembrenone, rolipram, ibudilast, roflumilast, apremilast, cisaborole, sildenafil, tadalafil, vardenafil, udenafil, avanafil, dipyridamole, quinazoline, paraxanthine, papaverine, a pharmaceutically acceptable salt of any of these, or any combination thereof.

In some embodiments, active pharmaceutical ingredients or salts thereof can comprise an antibiotic, an antiviral, an antiparasitic, a diuretic, a blood pressure medication, a phosphodiesterase inhibitor, a pharmaceutically acceptable salt of any of these, or any combination thereof. In some cases, an active pharmaceutical ingredient can comprise a hemp derived cannabinoid. beta-blocker, a nonsteroidal anti-inflammatory drug (NSAID), an ACE inhibitor, an angiotensin II receptor blocker, a calcium channel blocker, an alpha blocker, a cancer chemotherapeutic, a steroid, an immunomodulator, a pharmaceutically acceptable salt of any of these, or any combination thereof. In some cases, a cancer chemotherapeutic can comprise letrozole, sonidegib, ruxolitinib, abiraterone, altretamine, palbociclib, procarbazine, sunitinib, capecitabine, erlotinib, temozolomide, imatinib, lapatinib, sorafenib, rheumatrex, anagrelide, bexarotene, idelalisib, ixazomib, niraparib, olaparib, rucaparib, panobinostat, tegafur, gimeracil, oteracil, trifluridine, tipiracil, venetoclax, vinorelbine, vismodegib, lenalidomide, pomalidomide, thalidomide, abiraterone, apalutamide, enzalutamide, a salt thereof, or any combination thereof. In some cases, an antibiotic can comprise a penicillin, phenoxymethylpenicillin, flucloxacillin, cephalosporin, cefixime, cefpodoxime, cefuroxime, cephalexin, tetracycline, doxycycline, minocycline, an aminoglycoside, amoxicillin, ampicillin, a macrolide, azithromycin, gentamicin, tobramycin, clarithromycin, erythromycin, ciprofloxacin, levofloxacin, moxifloxacin, sulfacetamide, sulfadiazine, sulfamethoxazole-trimethoprim, clindamycin, a sulfonamide, a trimethoprim, a metronidazole, a quinolone, or a nitrofurantoin. An antiviral can comprise an acyclovir, peramivir, ganciclovir, valganciclovir, foscarnet, zanamivir, cidofovir, fomivirsen, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasbuvir, sofosbuvir, oseltamivir phosphate, remdesivir, balozavir marboxil, amantadine, rimantadine, baloxavir marboxil, a salt of any of these or any combination thereof. In some cases, an antiviral can comprise a Nucleoside Reverse Transcriptase Inhibitor (NRTI), a Non-Nucleoside Reverse Transcriptase Inhibitor (NNRTI), a Protease Inhibitor (PI), a fusion inhibitor, a CCR5 antagonist, an attachment inhibitor, a Integrase Strand Transfer Inhibitor (INSTI), a post attachment inhibitor, or a combination thereof. In some cases, an antiviral can comprise abacavir, dolutegravir, lamivudine, bictegravir, emtricitabine, tenofovir alafenamide, darunavir, cobicistat or a combination thereof. In some cases, an active pharmaceutical ingredient or salt thereof can comprise a potassium channel blocker such as dalfampridine or a salt thereof. In some cases, an active pharmaceutical ingredient or salt thereof can comprise levodopa, carbidopa, or a salt thereof. In some cases, an active pharmaceutical ingredient or salt thereof can comprise an antibody such as rHlgM22. In some cases, an active pharmaceutical ingredient or salt thereof can comprise a biologic such as GGF2 (Cimaglermin alfa). In some cases, an active pharmaceutical ingredient or salt thereof can comprise zolmitriptan or a salt thereof. In some cases, active pharmaceutical ingredients or salts thereof can comprise beta blockers (β-blockers), calcium blockers, angiotensin converting enzyme inhibitors, angiotensin receptor blockers, Nebivolol, CYP3A4 inhibitors, ketoconazole (Nizoral), itraconazole (Sporanox), erythromycin, saquinavir, clarithromycin, HIV protease inhibitors, alpha-adrenergic blocking agents (α-blockers), salts thereof, or any combination thereof.

In some embodiments, the composition can further comprise another set of active ingredients or salts thereof. For example, a second, third, or fourth different set of active ingredients. In some embodiments, the additional active ingredients or salts thereof can be administered in parallel or consecutively to enhance the efficacy of the first set of active ingredients or salts thereof. In some cases, a set of active ingredients can comprise one or more active ingredients.

In some embodiments, a composition can further comprise: an additional set of active ingredients or salts thereof which can be administered in parallel or consecutively to enhance the efficacy of cannabinoids or salt thereof. For example, the second different set of active ingredients or salts administered in parallel or consecutively to a cannabinoid can be a cytochrome P450 inhibitor. In some embodiments, a composition can comprise two or more different sets of active ingredients or salts thereof which can be administered in parallel or consecutively to enhance the efficacy of cannabinoids or salts thereof. In some embodiments, a composition can comprise 2, 3, 4, 5, 6, 7, 8, 9, 10 or more different sets of active ingredients.

In some embodiments the first set of active ingredients or salts can be administered in parallel or consecutively with a second different set of active ingredients. In some cases, an active ingredient can comprise dietary supplements. In some cases, an active ingredient can comprise a vitamin. In some cases, a vitamin can comprise vitamin A, vitamin B1, vitamin B2, vitamin B3, vitamin B5, vitamin B6, vitamin B7, vitamin B9, vitamin B12, vitamin C, vitamin D, vitamin E, vitamin K, a derivative of any of these, a salt of any of these, or any combination thereof. In some cases, an active ingredient can comprise an herbal supplement, for example, gingko, ginseng, fish oil, green tea, devil's claw, carnitine, chondroitin, coenzyme q, melatonin, soy, fenugreek, garlic or any combination thereof. In some cases, an active ingredient can comprise a mineral such as calcium, phosphorus, potassium, sodium, chloride, magnesium, iron, zinc, iodine, chromium, copper, fluoride, molybdenum, manganese, selenium or any combination thereof. In some cases, an active ingredient can comprise an amino acid, a lipid, a protein, or any combination thereof. In some cases, an amino acid can comprise histidine (H), arginine (R), alanine (A), isoleucine (I), cysteine (C), aspartic acid (D), leucine (L), glutamine (Q), asparagine (N), lysine (K), glycine (G), glutamic acid (E), methionine (M), proline (P), serine (S), phenylalanine (F), tyrosine (Y), selenocysteine (U), threonine (T), pyrrolysine (0), tryptophan (W), valine (V), a derivative of any of these, a salt of any of these or any combination thereof. In some cases, an active ingredient can comprise nitrates, nitric oxide, nitric oxide generating components, nitrite salts, nitrate salts, sodium nitrates, potassium nitrates, ascorbic acid, L-arginine, L-citrulline, magnesium ascorbate, sodium ascorbate, potassium ascorbate, salts thereof, or any combination thereof. In some cases, an active ingredient can comprise antihypertensive agents, diuretics, salts thereof, or any combination thereof.

In some embodiments, a second different set of active ingredients or salts may not be comprised in an oil composition. In some embodiments, a second different set of active ingredients or salts not comprised in the oil composition can be administered concurrently, in parallel, or consecutively.

In some embodiments, the composition has metabolites that can be pharmacologically active, retaining, at least partially, the potency of the parent pharmaceutical ingredient or the parent supplement ingredient.

In some embodiments, the composition comprising the salt of the active pharmaceutical ingredient or the active dietary supplement ingredient, can comprise an organic salt, an inorganic salt, or any combination thereof. In some cases, an organic salt can comprise a phosphinate (e.g. sodium hypophosphite), a hydrazinium salt, a urate, a diazonium salt, an oxalate salt, a tartrate, a choline chloride. An example of an inorganic salt can be sodium chloride, calcium chloride, magnesium chloride, sodium bicarbonate, potassium chloride, sodium sulfate, calcium carbonate, calcium phosphate, or any combination thereof.

In some embodiments, the pharmaceutical or dietary supplement composition comprising the salt of the active ingredient, wherein the salt can comprise an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof.

In some embodiments, a salt can comprise a pharmaceutically acceptable salt. In some embodiments, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

### Excipients and Other Non-Active Ingredients

In some embodiments, the composition described herein can comprise a pharmaceutically acceptable excipient. In some embodiments, a composition described herein can comprise an acceptable excipient for dietary supplements. In some embodiments, the composition can comprise an excipient. In some embodiments, the composition can comprise a pharmaceutically acceptable: carrier, diluent, or excipient. In some embodiments, the composition can comprise an acceptable: carrier, diluent, or excipient for a dietary supplement. In some cases, an excipient can comprise a carrier, diluent, or both. As used herein, “excipient” can refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some embodiments, an excipient can comprise anhydrous calcium phosphate, dihydrate calcium phosphate, a hydroxypropyl methylcellulose, a croscarmellose sodium, a GMO-free croscarmellose sodium, a carbomer, a magnesium aluminometasilicate, a mannitol, povidone (PVP), a crospovidone, a sorbitol, a dimethicone, a sodium stearyl fumarate, a sodium starch glycollate, a hydroxypropylcellulose, a native corn starch, a modified corn starch, a carrageenan, a alginate, a silicon dioxide, a microcrystalline cellulose, a carboxymethylcellulose sodium, a alginate, a carboxymethylcellulose (CMC), a sodium carboxymethylcellulose (Na CMC), a carbomer, a natural gum, a sorbitol, a maltitol, a glucose syrup, a silicone, a fatty alcohol, an alcohol, a carbohydrate, a petrolatum derivative, a butter, a waxe, DMSO Procipient®, an ester, a fatty acid, an oil-in-water (O/W) emulsifier, a water-in-oil (W/O) emulsifier, a silica, a fumed silica, a polysorbate, an isopropyl myristate, a cellulosic derivate, a xanthan gum, a propylenglycol, a noveon AA-1 polycarbophyl, a dimethyl isosorbate, polysilicone elastomer 1100, polysilicone elastomer 1148P, a preservative, a flavor, a color, a functional coating, an aesthetic coating, a pharmaceutically acceptable salt of any of these, or any combination thereof.

In some embodiments, an excipient can comprise a honey, a glycerin, an oil, a long chain sugar, a polyethylene glycol, a corn syrup, a maltodextrin syrup, a syrup, a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, an excipient can comprise honey. In some cases, a honey can comprise a clover honey, a buckwheat honey, an acacia honey, a manuka honey, a wildflower honey, a tupelo honey, an orange blossom honey, a sourwood honey, a linden honey, a sage honey, an eucalyptus honey, an avocado honey, a blueberry honey, a dandelion honey, a fireweed honey, a heather honey, a macadamia nut honey, wildflower honey, a palmetto honey or any combination thereof. In some cases, a honey can comprise a medical grade honey such as a medical grade manuka (Leptospermum scoparium) honey. In some embodiments, an excipient can comprise an oil, and an oil can comprise a food oil. In some cases, an excipient (e.g. honey) can at least partially protect an active ingredient from oxidation, degradation, microbial contamination or any combination thereof. In some cases, an excipient can comprise water and a sugar in the form of a syrup. In some cases, a sugar can comprise a granulated sugar, a confectioner's sugar, a fruit sugar, a superfine sugar, a cane sugar, a course sugar, a brown sugar, a white sugar, a muscovado sugar, a turbinado sugar, or a liquid sugar. In some cases, a sugar can comprise fructose, galactose, glucose, lactose, maltose, sucrose, xylose or any combination thereof. In some embodiments a food oil can comprise Medium-chain triglycerides (MCTs), long-chain triglycerides, an almond oil, an avocado oil, a brazil nut oil, a canola oil, a cashew oil, a chia seed oil, a cocoa butter oil, a coconut oil, a corn oil, a cottonseed oil, a flaxseed/linseed oil, a grape seed oil, a hemp seed oil, a vigna mungo oil, a mustard oil, an olive oil, a bean oil, a palm oil, a peanut oil, a pecan oil, a perilla oil, a rice bran oil, a safflower oil, a sesame oil, a soybean oil, a soybean oil, a walnut oil, a fish oil, an animal oil, a sunflower oil, a cottonseed oil, a vegetable oil or any combination thereof. In some embodiments, an excipient can comprise a carbohydrate. In some instances, the carbohydrate can comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. In some embodiments, an excipient can comprise lactose. In some instances, lactose can comprise a milled lactose, a sieved lactose, a micronized lactose, a spray dried lactose, an anhydrous lactose, a monohydrate lactose, or a combination thereof.

In some embodiments, an excipient can comprise a pharmaceutically acceptable excipient. In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof

In some embodiments, the active ingredient or pharmaceutically acceptable salt thereof can be contained at least in part within an excipient. In some embodiments, the active ingredient or pharmaceutically acceptable salt thereof can be contained at least in part in an excipient. In some embodiments, the active ingredient can be contained within a pore of an excipient. The “pore” of the excipient can refer to excipient particles that have been engineered to have open or closed pore structures. Porous excipient particles may be carriers of pharmaceutically active ingredients. Porous excipient particles may have a large surface area, stable structure, adjustable pore sizes, tunable dissolution, diffusion, or distribution, and well-defined surface properties. Porous excipient particles may facilitate sustained-release unit doses.

In some embodiments, in addition to the active pharmaceutical ingredients or salts thereof, the compositions can further comprise inactive ingredients selected from the group consisting of a microcrystalline cellulose, a anhydrous dibasic calcium phosphate, a croscarmellose sodium, a magnesium stearate, a hypromellose, a titanium dioxide, a lactose, a triacetin, a mannitol, a xylitol, a sorbitol, a sugar alcohols, a cellulose, a cellulose ester, a cellulose ether, a modified cellulose, a starch, a modified starch, a polysaccharide, an oligosaccharide, a disaccharide, a saccharide, a gelatin, a polyvinylpyrrolidone, a polyethylene a glycol, a binder, a flavorans, a colorant, FD & C Blue #2 aluminum lake, a magnesium stearate, a antiadherent agent, a stearate salt, a sweetener, a silica, a lubricant, or any combination thereof.

### Methods of Making Compositions

In some cases, methods of making a composition (e.g. a pharmaceutical composition or dietary supplement) can comprise creating particles by the methods described herein. In some cases, particles can comprise an excipient, an active ingredient, or both. In some cases, particles can comprise a carrier, an active ingredient, or both. In some cases, particles can comprise a diluent, an active ingredient, or both. In some embodiments, a method of making a composition, can comprise mixing, in a mixer, an excipient; and particles comprising an active ingredient or a pharmaceutically acceptable salt thereof at least partially encapsulated by a coating material and wherein the particles at least partially encapsulated by the coating material are spray dried. In some embodiments, a method of making a composition, can comprise formulating a capsule-in-capsule formulation in unit dose form. In some cases, the formulation can comprise particles comprising an active ingredient, or a pharmaceutically acceptable salt thereof. In some instances, the particles can be at least partially encapsulated by a coating material. In some instances, the particles at least partially encapsulated by the coating material can be spray dried. In some cases, the particles can be at least partially surrounded by a first capsule, a second capsule, or both. In some instances, the first capsule can be surrounded by a second capsule to create a capsule-in-capsule, capsule. In some cases, a capsule can comprise a capsule coating. In some cases, a capsule coating can at least partially control capsule ingredient release.

In some embodiments, a final product can be a capsule-in-capsule. In some cases, the final product can be a capsule (e.g. a second capsule) that surrounds an active ingredient and separately an inner capsule (e.g. the first capsule), which can contain its own active ingredient. In some instances, a capsule can contain more than one active ingredient. In some instances, a capsule can contain more than one inner capsule. For example, a capsule can comprise 1, 2, 3, 4, 5, 6, 7, 8, 9, 10 or more inner capsules. In some cases, an inner capsule can comprise a capsule. For example, an outer capsule can surround a first inner capsule and the first inner capsule can surround a second inner capsule.

The process described herein can include the following manufacturing stages. The active ingredient of the first capsule and the second capsule can be microencapsulated and spray dried using the methods described herein. In some embodiments, the active ingredients can be independently blended with an excipient or without an excipient. In some cases, the active ingredient of the first capsule can then be added to the first capsule and the first capsule can be banded using the methods described herein. In some cases, after the first capsule is banded, a capsule coating (e.g. an enteric, pH dependent, time release, or combination release) can be applied to the first capsule. In some cases, the active ingredient of the second capsule can then be added to the second capsule and the first capsule can be placed into the second capsule. The second capsule can be banded, and a capsule coating can be applied to the second capsule.

In some cases, a composition can comprise a mixture of particles described herein. In some instances, at least a portion of an excipient and at least a portion of the particles comprising an active ingredient, can comprise a mixture or a formulation. For example, spray dried encapsulated particles of a cannabinoid described herein can be mixed with a honey or a syrup.

In some embodiments, a method of making a composition can comprise the following steps: microencapsulation of an active pharmaceutical ingredient; spray drying, atomization and dry powder collection, blending of active pharmaceutical ingredient with an excipient, encapsulation into a first capsule, microencapsulation of a second active pharmaceutical ingredient, spray drying, atomization and dry powder collection, blending of the second active pharmaceutical ingredient with an excipient, encapsulation into a second capsule, and encapsulation of the first capsule into the second capsule.

### Microencapsulation

In some embodiments, encapsulation can comprise microencapsulation. Microencapsulation can be a process in which a microcapsule can be created as a small sphere or multi-sphere with a core and a matrix wall around it. The pharmaceutical ingredient inside the microcapsule can be called a fill. In some cases, a fill can be a liquid, an oil, a solid or any combination thereof. The wall around the fill (“or core”) can be referred to as a shell, a coating, or a membrane. In some cases, a microcapsule can have a diameter of about 50.0 microns to about 150.0 microns in size. In some instances, microcapsules can have a diameter as small as 1.0 micron in size to about 10.0 micron in size or about 1.0 micron to about 5.0 microns in size. In some cases, the small size can provide a pharmaceutical ingredient a large surface area. In some cases, microencapsulation can at least partially prevent administration of an active ingredient comprising the form of an unencapsulated crystal. For example, microencapsulation can at least partially prevent administration of unencapsulated crystals comprising an active ingredient or salt thereof. In some instances, unencapsulated crystals of an active ingredient can cause irritation of gastrointestinal tract. The irritation can be caused by crystal geometry and structure. For example, a crystal can have sharp angles and edges that can cause irritation, damage or both of the gastrointestinal tract during administration. In some instances, crystal geometry and structure can be controlled by the spray drying process. Microencapsulation can generate crystals with amorphous structure. In some instances, an amorphous crystal can lack sharp edges and angles. In some cases, an amorphous crystal can have a rounded edge. In some instances, an amorphous crystal may not cause irritation, damage or both of the gastrointestinal tract during administration.

In some cases, a cannabinoid such as CBD or a salt thereof in oil formulation or other active ingredients can be microencapsulated with compatible diluents to protect the oil from oxidation and provide a longer shelf life than the unprotected composition. The diluents can be aqueous, or solvent based and use animal or plant materials. In some cases, the diluent can comprise alcohols: e.g., ethanol, butanol, 2-ethylhexanol, isobutanol, isopropanol, methanol, propanol, propylene glycol; ketones: e.g., acetone, methyl ethyl ketone, methyl isobutyl ketone, methyl isopropyl ketone, mesityl oxide, trichloroethylene; halogenated solvents: e.g., ethylene bromide, chloroform, ethylene chloride, dichloromethane, tetrachloroethylene, carbon tetrachloride; amides: e.g., dimethylformamide; ethers: e.g., 1,4-dioxane, butyl ether, ethyl ether, di-isopropyl ether, tetrahydrofuran, tert-butyl methyl ether; sulfur containing solvent: e.g., dimethyl sulfoxide; amines: e.g., pyridine; nitriles: e.g., acetonitrile; esters: e.g., ethyl acetate; aliphatic hydrocarbons: e.g., cyclohexane hexane; aromatic hydrocarbons: e.g., toluene xylene; water or any combinations thereof. In some cases, the diluent can comprise benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, n-methylpyrrolidone, nitromethane, pyridine, sulfolane, tetralin, toluene, 1,1,2-trichloroethylene, xylene or any combinations thereof.

After the microencapsulation process, the suspension can be spray dried to create the dry powder finished product. In some cases, the microencapsulated powder can adapt well with small to large proteins and can be readily accepted in the body due to the permeable, large absorptive surface area in the gastrointestinal tract.

The core active ingredient can be microencapsulated with an amphipathic molecule that has both a polar end (‘hydrophilic”) and non-polar end (“hydrophobic”). In some cases, a hydrophilic end of an amphipathic molecule may interact with core material. In some cases, a hydrophobic end of an amphipathic molecule may interact with core material. This hydrophilic and hydrophobic structure can enable the molecule to microencapsulate an active ingredient and form a microsphere. In some instances, the microencapsulated particle may have a hydrophilic exterior and a hydrophobic interior. In some instances, the microencapsulated particle may have a hydrophobic exterior and a hydrophilic interior. The microencapsulation process can coat the active ingredient, which is the core, by the amphipathic encapsulating agent, which is the wall material, so that the active ingredient is at least partially surrounded by a wall of the amphipathic material. For example, hydroxypropyl methylcellulose acetate succinate (HPMCAS) can be an amphipathic molecule used to coat sildenafil or a salt thereof. The microencapsulation blend can be a spray dried dispersion, that can be fed into a spray dry system to create a hard-outer coating on the microcapsules.

The wall material can form a film that is cohesive with the core active ingredient. A wide variety of coating materials are available for encapsulation, e.g., traditional coating materials like inert polymers and pH sensitive ones as carboxylate and amino derivatives, which swell or dissolve according to the degree of cross-linking; some innovative coating polymers have also been developed for applications particularly among the bioadhesives and mucoadhesives. In some cases, the coating material can be hydrophilic polymers, hydrophobic polymers or a combination of both. In some cases, a microcapsule shell can comprise an amphipathic molecule. In some cases, the coating material can be gelatin, polyvinyl alcohol, ethyl cellulose, cellulose acetate phthalate and styrene maleic anhydride. In some instances, the coating material may not react with the pharmaceutical ingredient. In some cases, a microcapsule shell can comprise Hydroxypropyl methylcellulose (“HPMC”), Hydroxypropyl methylcellulose Acetate Succinate (“HPMCAS”), a cyclodextrin, maltodextrin, povidone, and others. In some instances, a microcapsule shell can comprise HPMCAS-LG, HPMCAS-MG, HPMCAS-HG or HPMC-P or a combination thereof. In some instances, a microcapsule shell can comprise a different grade of HPMC or HPMCAS. For example, a microcapsule shell can comprise an E5, an E50, or a K4M grade of HPMC. In another example, a microcapsule shell can comprise HPMCAS 716, HPMCAS 912, and HPMCAS 126 (the numbers referring to the ratio of succinyl and acetyl substituents). In another example, a microcapsule shell can comprise a L, a M, or an H grade of HPMCAS. In some cases, a microcapsule shell can comprise gelatin, cornstarch, polyvinylpyrrolidone (PVP), an oligosaccharide, a long chain sugar or any combination thereof. In some cases, a microcapsule shell can comprise a fatty acid, a liposome, an amino acid, a natural oil and a sugars, trehalose, dextran, a natural oil, a synthetic oil or a combination thereof. In some instances, an amino acid can comprise glutamic acid, aspartic acid, lysine, tryptophan, tyrosine, methionine or a combination thereof. In some cases, a fatty acid can comprise a polyunsaturated fatty acid, an essential fatty acid, a conjugated fatty acid, a short chain fatty acid, a medium chain fatty acid, a long chain fatty acid, a very long chain fatty acid, a saturated fatty acid, an unsaturated fatty acid, a monounsaturated fat, or any combination thereof. In some cases, a fatty acid can comprise an omega-3, an omega-5 fatty acid, an omega-6, an omega-7 fatty acid, an omega-9 fatty acid, an omega-10 fatty acid, an omega-11 fatty acid, an omega-12 fatty acid, or a combination thereof. In some cases, a natural oil can comprise soybean oil, a vegetable oil, a food oil, evening primrose oil, borage oil, blackcurrant seed oil, flax or linseed oil, rapeseed or canola oil, corn oil, almond oil, avocado oil, brazil nut oil, canola oil, cashew oil oil, chia seed oil, cocoa butter oil, coconut oil, corn oil, cottonseed oil, flaxseed/linseed oil, grape seed oil, hemp seed oil, vigna mungo oil, mustard oil, olive oil, palm oil, peanut oil, pecan oil, perilla oil, rice bran oil, safflower oil, sesame oil, soybean oil, walnut oil, sunflower oil, cottonseed oil, palm oil, or a combination thereof. In some cases, a microcapsule shell can increase or decrease active ingredient release kinetics or increase bioavailability. In some cases, encapsulation of a cannabinoid, a salt thereof, or an ester thereof and an excipient can produce about: 5% to about 70%, 5% to about 10%, 5% to about 20%, 10% to about 30%, 15% to about 40%, 25% to about 40%, 10% to about 60%, or 20% to about 50% more bioavailability of the cannabinoid, the salt thereof, or the ester thereof as compared to a cannabinoid, a salt thereof, or an ester thereof that is not encapsulated when administered to a subject. The wall material can be biodegradable and biocompatible with the pharmaceutical ingredient. In some cases, a microcapsule can be produced by dissolving or mixing the pharmaceutical ingredient in a solvent with the shell material to produce a liquid suspension. For example, HPMCAS can be dissolved with ethanol and water and a pharmaceutical compound can be added the liquid suspension. In some instances, the pharmaceutical compound may not dissolve in the liquid suspension. In some instances, the pharmaceutical compound may dissolve in the liquid suspension. The liquid suspension can be dried with a spray drying technique described herein or another method.

In some cases, the average wall thickness can of a microencapsulated particle can be about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness can of a microencapsulated particle can range from about: 1 μm to about 10 μm, 1 μm to about 5 μm, 2 μm to about 7 μm, 3 μm to about 8 μm, 5 μm to about 10 um, 5 μm to about 15 um, or 1 μm to 30 um. In some instances, the wall thickness of a microencapsulated particle can increase by increasing the ratio of the wall material to the core material prior to spray drying. In some cases, the ratio of wall material to core material (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material to core material (weight/weight) can be about 10:1.

The core material can be the material over which coating has to be applied to serve the specific purpose. Core material may be in form of solids or droplets of liquids and dispersions. In some cases, core material can comprise sildenafil, a salt thereof, or an ester thereof. In some cases, core material can comprise a cannabinoid or a salt thereof. Core material can be an oil. The composition of core material can vary and thus furnish definite flexibility and allow effectual design and development of the desired microcapsule properties. A substance may be encapsulated for a number of reasons. Examples may include protection of reactive material from their environment, safe and convenient handling of the materials which can be otherwise toxic or noxious, taste masking, means for controlled or modified release properties means of handling liquids as solids, preparation of free flow powders and in modification of physical properties of the drug. For example, encapsulation can improve solubility and dissolution and therefore increase and enhance bioavailability of an active ingredient such as sildenafil, a salt thereof, an ester thereof, or a cannabinoid, or a salt thereof. Microencapsulation can be used to increase the stability, improve the handling properties of compounds, facilitate higher bioavailability when reconstituted or administered, or any combination thereof.

In some cases, microencapsulation of a cannabinoid or a salt thereof by HPMCAS can provide faster absorption in the gastrointestinal tract. For example, THC may not be water soluble and microencapsulation with HPMCAS can provide increased absorption into the blood stream from the gastrointestinal tract. In some instances, microencapsulation can increase the solubility of an active ingredient. In some cases, a microencapsulated cannabinoid or a salt thereof may be absorbed about: 10% to about 70%, 5% to about 10%, 5% to about 20%, 10% to about 30%, 15% to about 40%, 25% to about 40%, 35% to about 50%, 10% to about 60%, 40% to about 90%, or 20% to about 50% faster than a cannabinoid that is not microencapsulated.

In some embodiments, a method of microencapsulation can comprise at least partially dissolving the coating material (e.g. HPMC or HPMCAS) in a solvent such as an ethanol and water mix. In some cases, a cannabinoid oil or salt thereof can be micronized with a micronizer to generate small oil droplets. In some cases, a microfluidic system can be used to generate small oil droplets. The oil droplets can be added to the solution of the coating material and the solvent to create a suspension of the oil droplets and the coating material dissolved in the solvent. In some instances, the oil droplets may not dissolve in the suspension and may remain in suspension. The suspension can be mixed to an at least partially uniform mixture and spray dried. The coating can at least partially encapsulate the oil droplets containing the cannabinoid or salt thereof. In some cases, the encapsulation of a cannabinoid can be a spherical, round, oval, or any shape structure.

### Spray Drying, Atomization and Dry Powder Collection

In some embodiments, a method of making a composition can comprise particles wherein at least a portion of the particles of the active ingredient or a pharmaceutically acceptable salt thereof can be made by a spray drying process.

In some embodiments, the spray drying process can comprise: atomizing liquid droplets comprising the active ingredient or the pharmaceutically acceptable salt thereof, drying the droplets from particles, recovering the particles, or any combination thereof. In some cases, the liquid droplets can comprise an encapsulated active ingredient.

In some embodiments, a spray drying manufacturing system can comprise a closed spray dryer container which receives the solution comprising a drug dissolved in a suitable solvent (aqueous or solvent based). In some cases, a solvent can comprise alcohol, ethanol, dimethylformamide (DMF), dimethyl sulfoxide (DMSO), a polar organic solvent, an organic solvent, or any combination thereof. In some embodiments, the solution then enters the particle formation chamber which can be connected to an atomizer located at the top of the chamber. In some cases, the atomizer can use a gas. In some embodiments, the atomizer can be a two component or rotary nozzle type that distributes the solution into fine droplets controlled by the atomizer pressure. In some embodiments, the atomizer can be a rotary atomizer that employ an atomizer wheel rotating at high speed.

In some embodiments, this atomization gas can be an inert gas. As used herein, “inert gas” can refer to a non-reactive gas, or a gas that does not undergo chemical reactions under a set of given conditions. Inert gases can be generally used to avoid unwanted chemical reactions degrading a sample, or to prevent bacterial growth. These undesirable chemical reactions can often be oxidation and hydrolysis reactions with the oxygen and moisture in air. The term “inert gas” can be context-dependent because several of the noble gases, which have been historically referred to as the inert gases, can be made to react under certain conditions. In some embodiments, inert gas can be air, nitrogen, carbon dioxide or any combination thereof. In some embodiments, the atomized droplets go through a hot gas drying chamber to produce uniform fine particles that maintain a tight particle size distribution following liquid evaporation. In some cases, the solid particle forms and falls to the bottom of the drying chamber. In some instances, the balance between temperature, flow rate, and droplet size can controls the drying process. In some embodiments, the powder can be recovered from the exhaust gas using a cyclone separator or a bag filter.

The moisture level of the powder after spray drying can be below about 10%. In some embodiments, the moisture level can be below about 15%, about 14%, about 13%, about 12%, about 11%, about 10%, about 9%, about 8%, about 7%, about 6%, about 5%, about 4%, about 3%, about 2%, or about 1%.

Blending of Active Pharmaceutical Ingredient with Excipient

In some embodiments, a particle size can be validated by a particle analyzer (e.g. a Malvern particle analyzer) prior to blending with an excipient. In some embodiments, the active powder can be blended with an excipient carrier (e.g., honey or glycerin) product in a blender or mixer and the blended composition can be fed to a hopper or another loading device.

### Methods of Making the Composition to Select Sizes

In some embodiments, a method of making a pharmaceutical or dietary supplement can comprise mixing an excipient comprising an active ingredient or a pharmaceutically acceptable salt thereof at least partially encapsulated by a coating material and wherein the particles at least partially encapsulated by the coating material are spray dried. In some embodiments, a method of making a composition can comprise mixing ingredients in a mixer. In some embodiments, a method of making a composition can comprise mixing particles of one or more active ingredients in a mixer. In some embodiments, a method of making a composition can comprise formulating a capsule-in-capsule formulation in unit dose form. In some cases, a method of formulating a capsule-in-capsule formulation can comprise formulating the particles comprising an active ingredient, or a pharmaceutically acceptable salt thereof. In some cases, the particles can be at least partially encapsulated by a coating material. In some cases, the particles may not be at least partially encapsulated by a coating material. For example, a coating material may not be added to an active ingredient. In some instances, the particles at least partially encapsulated by the coating material can be spray dried. In some cases, the particles can be at least partially surrounded by a first capsule, a second capsule, or both. In some cases, the first capsule is surrounded by a second capsule. In some cases, the capsules can comprise a capsule coating to at least partially control active ingredient release.

In some embodiments, the method of making a composition can comprise formulating the particles described herein into a capsule-in-capsule composition. In some instances, at least a portion of the particles comprising an active ingredient or a pharmaceutically acceptable salt thereof at least partially encapsulated by a coating material can have a particle diameter ranging from about 30.0 micrometers to about 200 micrometers or about 50.0 micrometers to about 150.0 micrometers, as measured by a particle size analyzer using laser diffraction. In some instances, at least a portion of the particles comprising an active ingredient or a pharmaceutically acceptable salt thereof can have a particle diameter ranging from about 30.0 micrometers to about 200 micrometers or about 50.0 micrometers to about 150.0 micrometers, as measured by a particle size analyzer using laser diffraction.

In some embodiments, at least a portion of the particles of an excipient can have a particle diameter ranging from about: 30 μm (micrometers) to about 60 μm, 50 μm to about 200 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, particles of an excipient can have a particle diameter of more than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, particles of an excipient can have a particle diameter of less than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm.

In some embodiments, particles comprising an active ingredient or a pharmaceutically acceptable salt thereof at least partially encapsulated by a coating material can have particle diameters ranging from about: 20 μm (micrometers) to about 60 μm, 30 μm to about 200 μm, 50 μm to about 150 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, particles comprising an active ingredient or a pharmaceutically acceptable salt thereof at least partially encapsulated by a coating material can have a particle diameter of less than about: 20 μm, 30 μm, 40 μm, 45 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 155 μm, 160 μm, 165 μm, 170 μm, 175 μm, 180 μm, 185 μm, 190 μm, 195 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, particles comprising an active ingredient or a pharmaceutically acceptable salt thereof at least partially encapsulated by a coating material can have a particle diameter of more than about: 20 μm, 30 μm, 40 μm, 45 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 155 μm, 160 μm, 165 μm, 170 μm, 175 μm, 180 μm, 185 μm, 190 μm, 195 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm.

In some embodiments, particles comprising an active ingredient or a pharmaceutically acceptable salt thereof can have particle diameters ranging from about: 20 μm (micrometers) to about 60 μm, 30 μm to about 200 μm, 50 μm to about 150 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, particles comprising an active ingredient or a pharmaceutically acceptable salt thereof can have a particle diameter of less than about: 20 μm, 30 μm, 40 μm, 45 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 155 μm, 160 μm, 165 μm, 170 μm, 175 μm, 180 μm, 185 μm, 190 μm, 195 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, particles comprising an active ingredient or a pharmaceutically acceptable salt thereof can have a particle diameter of more than about: 20 μm, 30 μm, 40 μm, 45 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 155 μm, 160 μm, 165 μm, 170 μm, 175 μm, 180 μm, 185 μm, 190 μm, 195 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm.

In some embodiments, particles of an active ingredient or a pharmaceutically acceptable salt thereof can be mixed in sizes. In some cases, the mixed sizes can change the release time of the drug. For example, encapsulated particles with small sizes can be absorbed faster into the blood stream while larger encapsulated particles can take longer to be absorbed into the blood stream. In some embodiments, the particles with larger sizes (about 100 μm to about 200 μm) can be mixed with particles with smaller sizes (about 30 μm to about 80 μm). In some embodiments, the weight to weight ratio of the particles with larger sizes (about 100 μm to about 150 μm) to the particles with smaller sizes (about 20 μm to about 80 μm) can be range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or about 1:8 to about 1:10.

In some embodiments, a particles or compositions described herein can have a tap density of more than about: 0.1 grams/centimeter³ (g/cm³), 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some embodiments, a particles described herein can have a tap density of less than about: 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some cases, particles or compositions described herein can have a tap density of more than about 0.6 g/cm³, 0.7 g/cm³. In some cases, tap density can be a measure of the envelope mass density characterizing a particle. The envelope mass density of a particle of a statistically isotropic shape can be defined as the mass of the particle divided by the minimum sphere envelope volume within which it can be enclosed. Features which can contribute to low tap density include irregular surface texture, porous structure or a combination thereof. Tap density can be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.).

In some embodiments, particles that are not of an excipient, can have particle diameters ranging from about 50 μm to about 150 In some embodiments, particle diameters can be measured by a particle analyzer using laser diffraction (LD), static light scattering, dynamic light scattering (DLS), or nanoparticle tracking analysis (NTA).

### Packaging of the Compositions

In some embodiments, a composition can be contained within a capsule, a tablet, a gummy, or any combination thereof. In some cases, a capsule can be a capsule-in-capsule.

In some embodiments, the capsule may comprise a single-piece capsule, a two-piece capsule, a transparent capsule, a non-transparent capsule, an opaque capsule, a slow-release capsule, an extended-release capsule, a standard-release capsule, a rapid-release capsule, a quick-release capsule, a hard-shell capsule, a soft gel capsule, a gel capsule, a hard gelatin capsule, a soft gelatin capsule, an animal-based capsule, a vegetarian capsule, a polysaccharide capsule, cellulose capsule, a mucopolysaccharide capsule, a tapioca capsule, a hydroxypropyl methyl cellulose (HPMC) capsule, a pullulan capsule, an enteric capsule, an uncoated capsule, a coated capsule, a capsule comprising titanium dioxide, fatty acids, waxes, shellac, plastics, plasticizers, glycerin, sorbitol, plant fibers, additives, preservatives, colorants, or any combination thereof. In some embodiments, the capsule may comprise a vegetarian capsule. In some embodiments, the capsule can comprise a capsule-in-capsule as described herein. For example, second capsule can comprise a first capsule.

In some embodiments, a capsule can further comprise a capsule coating. In some cases, a capsule coating can be added to a capsule to further improve stability (light protection, moisture and gas barrier), facilitate administration, or modify the drug release behavior from the dosage form. A capsule coating may be used to enable the immediate release of the drug, delay the release of the drug (such as in enteric coatings), or sustain the release of the drug from the dosage form over extended periods of time. In some instances, a capsule coating can comprise a film coating, a gelatin coating, or both. In some instances, a capsule coating can comprise an enteric coating, a time release coating, a pH dependent coating, a delayed release coating, an extended release coating, or a combination thereof. For example, an enteric coating can be added to a capsule to prevent it from dissolving until after it passes through the stomach. In some cases, the medication can release depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH coating can be used to dose areas of the GI tract with specific pH levels. In some embodiments, an enteric coating of a capsule can be a polymer barrier that can be applied to the capsules described herein to enable a controlled release. Bypassing the stomach can allow for more precise dosing and can enable the drug to achieve a higher bioavailability in the gastric track. In some cases, these coatings can be modified to deliver medicine from the mouth, all the way to the colon. In some cases, the technology can be applied to the outer (e.g. the second capsule) and the inner (e.g. the first capsule) capsule in the capsule-in-capsule technology and utilize time-released, pH-controlled released, or a combination of both technologies to achieve the intended drug delivery. In some instances, an enteric coating can be applied to multiple capsules, for example to an inner capsule and to an outer capsule and to provide delayed release of both capsules. In some cases, a capsule coating can provide a color, mask a bitter taste, or both. In some cases, a capsule coating can comprise polymers, plasticizers, pigments, opacifiers, glidants, binders, anti-tacking agents, anti-foaming mechanisms, surfactants, fillers, and extenders.

In some embodiments, a capsule can comprise a pH buffering composition to alter the pH of the environment of the capsule when released. In some instances, a pH buffering composition can comprise an excipient. In some cases, an excipient can comprise a pH buffering composition. In some instances, a pH buffering composition can comprise sodium phosphate, citric acid, acetic acid, potassium phosphate, tromethamine, gluconic acid, lactic acid, tartaric acid, aspartic acid, glutamic acid, citric acid cycle intermediates (Citrate, fumarate, a-ketoglutarate, malate and succinate) or any combination thereof.

In some embodiments, a capsule can be configured (for example with a capsule coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

In some embodiments, the capsule having different sizes according to composition requirements. In some embodiments, the capsule size can be: Su07, 7, 10, 11, 12e1, 12, 13, 000, 0E, 00, 0, 1, 2, 3, 4, or 5. In some embodiments, a capsule size can be 000. In some embodiments, a capsule size can be 00. In some embodiments, a capsule size can be 0. In some embodiments, a capsule size can be 1. In some embodiments, a capsule size can be 2. In some embodiments, a capsule size can be 3. In some embodiments, a capsule size can be 4. In some embodiments, a capsule size can be 5. In some embodiments, the capsule capacity varies from about 0.13 ml (e.g. size 5) to about 28 ml. In some embodiments, the capsule capacity varies from about 0.13 ml to about 1.37 ml. In some embodiments, a first capsule (e.g. the inner capsule) can be size: 000, 00, 0, 1, 2, 3, 4, or 5. In some embodiments, a second capsule (e.g. the outer capsule) can be size: 000, 00, 0, 1, 2, 3, or 4. In some cases, a capsule encapsulating another capsule will be different sizes. For example, an outer capsule can be larger (e.g. size 0 or size 00) than an inner capsule (e.g. size 1 or size 3) which it can encapsulate.

In some embodiments, the blended (or mixed) active ingredient and excipient can be loaded into an encapsulator machine “encapsulator”, which feeds the blend into a capsule. The encapsulator can automatically separate the capsule top “cap” and body “shell” and the powder can be slugged (liquids can be placed into the capsule with a piston type or rotary pump) and then transferred into the body of the capsule. The capsule halves can be then closed together to form an enclosed capsule that contains the blended powder. In some cases, during filling, the capsule atmosphere can be made inert with nitrogen to prevent oxidation and remove moisture from the blend so that the mixture of a liquid, a powder, or any combination thereof can stay dry. In some instances, the active ingredient can be placed into a first hypromellose capsule. In some cases, another capsule containing an active ingredient can comprise the first hypromellose capsule to create a capsule-in-capsule composition.

In some embodiments a capsule band can be added to a capsule. In some cases, capsule banding can be the process of sealing the capsule so that it may be filled with liquids, powders or other types of ingredients. In some instances, there can be a seal joint between the capsule cap and body that can require an additional band to be applied to prevent leakage of the drug outside the capsule. In some instances, it can provide a tamper resistant band that can reduce oxidation and minimizes any odor. In some cases, the banding can be applied with a banding machine that applies a thin layer of HPMC (hydroxypropyl methylcellulose) as the capsules pass over two rollers which apply the capsule banding material. In this process, the banding material can be heated and temperature controlled to make a smooth, liquid-tight band that join the capsule top and body. This can provide a visual tamper resistant barrier on the capsule.

In some cases, a capsule can have an oval shape, round shape, a square shape, an oval shape, a tube shape, an oblong shape, or a suppository shape. In some cases, the capsule can be any shape.

In some embodiments, the composition described herein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

In some embodiments, a composition can be contained within a capsule, wherein the capsule can be loaded with about 5% to about 99% (by volume) with the composition. In some cases, the capsule can be loaded with about: 5%, 10%, 15%, 20%, 25%, 30%, 31%, 32%, 33%, 34%, 35%, 36%, 37%, 38%, 39%, 40%, 45%, 50%, 55%, 60%, 65%, 70%, 75%, 80%, 85%, 90%, 95%, 99% (by volume) with a composition described herein. In some embodiments, the capsule can be loaded with about 5% to about 20%, about 20% to about 25%, about 20% to about 30%, about 20% to about 40%, about 20% to about 50%, about 20% to about 60%, about 20% to about 65%, about 20% to about 70%, about 20% to about 75%, 25% to about 30%, about 25% to about 40%, about 25% to about 50%, about 25% to about 60%, about 25% to about 65%, about 25% to about 70%, about 25% to about 75%, about 30% to about 40%, about 30% to about 50%, about 30% to about 60%, about 30% to about 65%, about 30% to about 70%, about 30% to about 75%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, about 70% to about 75%, or about 75% to about 100% (by volume) with the composition. In some cases, a capsule can be loaded with another capsule to create a capsule-in-capsule composition.

In some embodiments, the content of the capsule can comprise less than about: 10%, 9%, 8%, 7%, 6%, 5%, 4%, 3%, 2% or 1% water by weight. In some embodiments, the content of the capsule can comprise less than about 50%, about 40%, about 30%, about 25%, about 20%, about 10%, about 5%, or about 1% water by weight.

In some embodiments, the total content of all gases in the capsule can be less than about: 10%, 9%, 8%, 7%, 6%, 5%, 4%, 3%, 2% or 1% water by weight. In some embodiments, the total content of all gases in the capsule can be less than about 50%, about 40%, about 30%, about 25%, about 20%, about 10%, about 5%, or about 1% water by weight.

In some embodiments, the capsule can further comprise, in the volume not occupied by a composition, a capsule, or both an inert gas. In some embodiments, the inert gas can comprise an elemental gas, a compound gas, a noble gas, helium, neon, argon, krypton, xenon, oganesson, compounds of noble gas, purified argon, purified nitrogen, nitrogen or any combination thereof. In some embodiments, the inert gas can comprise nitrogen.

In some cases, the same active ingredients can be independently separated into the inner capsule, the outer capsule, or both of a capsule-in-capsule composition. In some cases, different active ingredients can be independently separated into the inner capsule, the outer capsule, or both of a capsule-in-capsule composition. For example, an active ingredient comprising sildenafil, a salt thereof, or an ester thereof can be comprised in in an inner capsule and the outer capsule can comprise a cannabinoid or a salt thereof such as CBD. In some cases, active compounds in the inner, outer or both capsules can comprise an excipient such as lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, honey, corn starch, carrageenan, sorbitol, maltitol, glucose, a pharmaceutically acceptable salt of any of these. In some cases, the active compounds can be microencapsulated as described herein. In some embodiments, the cannabinoid or a salt thereof, sildenafil, a salt thereof, or an ester thereof, or any combination thereof can be encapsulated in a coating material (e.g. HPMCAS) and can be spray dried (e.g. microencapsulated) as described herein. After the microencapsulation process, the suspension can be spray dried to create a dry powder finished product. In some cases, a dry powdered finished product can be added to a capsule. In some instances, sildenafil, a salt thereof, or an ester thereof can be comprised in a composition in an amount of about: 1000 μg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some cases, the inner and the outer capsule can have the same amount of an active drug. In some instances, the inner and the outer capsule may have a different amount of an active drug. In some cases, the ratio of the amount of the compound in the inner capsule and the outer capsule (weight to weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1 or 100:1. In some cases, the ratio of the amount of the compound in the inner capsule and the outer capsule (weight to weight) can be about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95 or 1:100.

In some cases, CBD, THC or both can comprise a cancer chemotherapeutic. In some cases, a cannabinoid can be administered as a cancer chemotherapeutic. In some instances, CBD, THC or both can cause apoptosis of a cancer cell. In some instances, CBD, THC or both can elicit anti-neoplastic effect. In some cases, a cannabinoid such as CBD or THC may bind to a receptor on a cancerous cell. In some cases, a cannabinoid can at least partially bind to a G-protein coupled CB-receptor such as CB1-R and CB2-R. For example, THC may be a partial agonist for CG1-R, CB2-R or both. In another example, CBD may be an inverse agonist for CB1-R, CB2-R or both. In some instances, cannabinoids can at least partially inhibit cell cycle progress, at least partially inhibit cell growth, at least partially induce apoptosis, or any combination thereof of cancer cells. In some cases, a cannabinoid can at least partially inhibit migration of a cancer cell. In some instances, a cannabinoid can at least partially inhibit angiogenesis of cancer cells. In some cases, one or more cannabinoids can be administered as a cancer chemotherapeutic. In some cases, a cannabinoid can be administered in an amount of about: 1 mg to about 5 mg, 1 mg to about 10 mg, 10 mg to about 15 mg, 5 mg to about 20 mg, 1 mg to about 200 mg, 2 mg to about 50 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, or 300 mg to about 600 mg as a cancer chemotherapeutic.

In some embodiments, an active ingredient can comprise a cancer chemotherapeutic. For example, letrozole, sonidegib, ruxolitinib, abiraterone, altretamine, palbociclib, procarbazine, sunitinib, capecitabine, erlotinib, temozolomide, imatinib, lapatinib, sorafenib, rheumatrex, anagrelide, bexarotene, idelalisib, ixazomib, niraparib, olaparib, rucaparib, panobinostat, tegafur, gimeracil, oteracil, trifluridine, tipiracil, venetoclax, vinorelbine, vismodegib, lenalidomide, pomalidomide, thalidomide, abiraterone, apalutamide, enzalutamide, a salt thereof, or any combination thereof, can be comprised in in an inner capsule and the outer capsule can comprise a cannabinoid or a salt thereof such as CBD. In some cases, a cancer chemotherapeutic can be in the inner capsule and a cannabinoid such as THC can be in the outer capsule. In some cases, the inner capsule, the outer capsule, or both can comprise microencapsulated compounds. In some instances, the inner capsule, the outer capsule, or both may not comprise microencapsulated compounds. In some cases, CBD can bind to a fatty acid binding protein that transport anandamide intracellularly to Fatty Acid Amide Hydrolase (FAAH) for degradation, which may play a role in the inhibition of anandamide metabolism by CBD. In some instances, CBD can reduce MAGL-mediated degradation of 2-AG. In some cases, a composition described herein such a cannabinoid (e.g. CBD or a salt thereof) can enhance the effect of another drug. For example, administration of CBD or a salt thereof can enhance the treatment of a cancer or increase the bioavailability of a drug. In some instances, CBD or a salt thereof can be a competitive inhibitor of cytochrome P450 and at least partially prevent cytochrome P450 from metabolizing other compounds. For example, administration of a capsule-in-capsule with CBD in the outer capsule and a cancer chemotherapeutic in the inner capsule may enhance the treatment of a cancer by increasing the bioavailability of a chemotherapeutic as P450 may be at least partially inhibited by CBD.

### Delivery of the Composition

In some embodiments, the administration of a composition (e.g. the pharmaceutical composition or a dietary supplement) or the second therapeutic can be administered orally, anally, vaginally, through the urethra, or any combination thereof. In some embodiments, the administration is by a capsule or a tablet. In some embodiments, a second therapeutic or a pharmaceutically acceptable salt thereof can be administered orally, intranasally, intraocularly, anally, by injection, intravenously, intramuscularly, subcutaneously, intraperitoneally, trans dermally, or any combination thereof.

In some embodiments, administering can be by oral ingestion, or by a suppository. In some cases, a suppository can comprise a rectal suppository, a vaginal suppository or a urethral suppository. In some embodiments, administering can comprise oral ingestion and the oral ingestion can comprise oral ingestion of a liquid, a capsule, a tablet, or any combination thereof. For example, a capsule described herein can be dissolved in a liquid prior to administration. In some cases, a capsule can be opened and applied to a food to be administered. In some cases, a capsule can be administered by placing a capsule in a food and administering the food. In some embodiments, administering can comprise oral ingestion of a capsule. In some embodiments, administering can comprise administration of a suppository capsule. In some embodiments, administering can be performed at least about: 1 time per day, 2 times per day, 3 times per day, 4 times per day, 5 times per day, 6 times per day or more than 6 times per day. In some cases, administering can be performed daily, weekly, monthly, or as needed. In some embodiments, administering can be conducted one, twice, three, or four times per day. In some cases, administration can be provided by a subject (e.g. the patient), a health care provider, a dietitian, a veterinarian, or a combination thereof.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years.

Also disclosed herein are kits comprising the pharmaceutical composition, the dietary supplement composition, or the composition for the non-human animal contained at least in part in packaging. Also disclosed herein are methods of making kits comprising a pharmaceutical composition, a dietary supplement composition, or a composition for a non-human animal contained at least in part in packaging.

Also disclosed herein are methods of treating or preventing a disease comprising treating or preventing the disease or condition by administering a therapeutically effective amount of a pharmaceutical composition. Also disclosed herein are methods of treating or preventing a disease comprising treating or preventing the disease or condition by administering, (e.g. via oral administration of a capsule) a therapeutically effective amount of the pharmaceutical composition. In some embodiments, the disease can comprise treating or preventing a disease or condition selected from the group consisting of: a cancer, an anxiety, pruritus (itching), cognitive function, Alzheimer's disease, an ulcerative colitis, irritable bowel syndrome, a chronic pain, pain management, multiple sclerosis, side effects of chemotherapy, AIDS, HIV, a neurodegenerative disorder, Tourette syndrome, cervical dystonia, a sleep aide, an appetite stimulant, a nausea associated with chemotherapy, a nausea, anorexia, spinal cord injury, glaucoma, an epilepsy, a seizure, an asthma, dependency withdrawal, a psychiatric symptom, an autoimmune disease, an inflammation, and any combination thereof. In some embodiments, a cancer can be a breast cancer, a brain cancer, a tumor, a cervical cancer, a lung cancer, a prostate cancer, a pancreatic cancer, or any combination thereof. In some cases, a cancer can be a sarcoma, a melanoma, a lymphoma, a leukemia, or a combination thereof. In some cases, a disease can comprise neuropathic pain, a heart disease, a brain tumor, a human papillomavirus (HPV) infection, a brain injury (e.g. a traumatic brain injury), a depression, inflammation, Huntington's Disease, emesis, osteoporosis, schizophrenia, a cardiovascular disease, obesity, an infectious disease (bacterial, fungal, or viral), a metabolic syndrome-related disease, an arthritis, fibromyalgia, a dementia, Parkinson's disease or any combination thereof. In some cases, an arthritis can comprise osteoarthritis, fibromyalgia, rheumatoid arthritis, psoriatic arthritis, juvenile arthritis, gout, lupus. In some cases, a disease or condition can comprise pain, such as a chronic pain or an acute pain associated with an arthritis. In some cases, a disease or condition can comprise a pain associated with HIV, such as a chronic pain, an acute pain, or both. In some cases, a disease or condition can comprise inflammation associated with HIV. In some cases, a disease can comprise sickle cell disease. In some instances, sickle cell disease can comprise sickle cell anemia, sickle hemoglobin-C disease, sickle beta-plus thalassemia or sickle beta-zero thalassemia. In some cases, a disease or condition can comprise a pain (e.g., an acute pain or a chronic pain) associated with sickle cell anemia, sickle hemoglobin-C disease, sickle beta-plus thalassemia or sickle beta-zero thalassemia. In some cases, a disease or condition can comprise inflammation associated with sickle cell anemia, sickle hemoglobin-C disease, sickle beta-plus thalassemia or sickle beta-zero thalassemia. In some cases, a composition described herein can alleviate symptoms associated with a disease. For example, a composition described herein can alleviate anemia, fatigue, pain, swelling (e.g., of hands and/or feet), infections, delayed growth, vision problems or any combination thereof. In some embodiments, the disease can comprise treating or preventing a disease or condition selected from the group consisting of: erectile dysfunction, a respiratory infection, COVID-19, a corona virus infection, a viral infection, a bacterial infection, a fungal infection, a parasitic infection, influenza, influenza type A, influenza type B, pulmonary arterial hypertension, heart disease, arrhythmia, cardiomyopathy, high blood pressure, sleep apnea, a headache, a migraine, an allergy, an autoimmune disease, Raynaud's disease, a cancer, asthma, chronic obstructive pulmonary disease, bronchitis, chronic bronchitis, a pneumonia, pulmonary edema, emphysema, pain, chronic pain, anxiety, opioid addiction, opioid overdose, increasing exercise performance, and any combination thereof. In some cases, a composition such as a cannabinoid can be administered as an antimicrobial, an anti-inflammatory, or both.

In some embodiments, prior to treating, a subject may have been diagnosed with the disease. In some embodiments, the subject may be a human, a man, a woman, an individual over 18 years of age, an individual under 18 years of age, or any combination thereof. In some embodiments, the subject can be an animal. In some cases, an animal can be a mammal, a reptile, an amphibian, or a bird. In some embodiments, a subject can be a pet or livestock. Non-human animals can include simians, a cat, a murine, a rat, a sheep, a lamb, a llama, a mouse, a chinchilla, a canine, a leporid, a rabbit, livestock, a sport animal, and a pet. In some embodiments, an animal can comprise a pet. In some cases, a pet can comprise a dog, a cat, a rodent, a hamster, a horse, a goat, a bird, a reptile, a pig, a cow, or a rabbit. In some embodiments, an animal can be about 1 day to about 10 months old, from about 9 months to about 24 months old, from about 1 year to about 8 years old, from about 5 years to about 25 years old, from about 20 years to about 50 years old, or from 40 years to about 120 years.

In some embodiments, a subject can be from about 1 day to about 10 months old, from about 9 months to about 24 months old, from about 1 year to about 4 years old, from about 3 years to about 10 years old, from about 8 years to about 15 years old, from about 10 years to about 20 years old, or from about 15 years to about 30 years old. In some embodiments, a subject can be about: 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years old.

In some embodiments, a method can further comprise diagnosing a subject as having the disease. In some embodiments, a diagnosing can comprise employing an in vitro diagnostic. In some embodiments, the in vitro diagnostic can be a companion diagnostic.

In some embodiments, a diagnosis can comprise a physical examination, a radiological image, a blood test, an antibody test, or any combination thereof. In some embodiments, a diagnosis can comprise a radiological image and the radiological image can comprise: a computed tomography (CT) image, an X-Ray image, a magnetic resonance image (MM), an ultrasound image, or any combination thereof.

In some embodiments, a method can further comprise administering a second therapy (e.g. a second therapeutic) to the subject. In some cases, a second therapy can be administered concurrently or consecutively to a pharmaceutical composition described herein. In some cases, a second therapeutic can be comprised in the pharmaceutical composition described herein. In some cases, a second therapeutic may not be comprised in the pharmaceutical composition described herein. In some embodiments, a second therapy can comprise acetaminophen, a corticosteroid, an opioid, a nonsteroidal anti-inflammatory drug (NSAID), a COX-2 selective NSAID, a COX-2 inhibitor, methotrexate, hydroxychloroquine, prednisone, cortisone, a biological response modifier, a salt thereof, or any combination thereof. In some embodiments, a second therapy can comprise a biological response modifier and the biological response modifier can comprise: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof. In some embodiments, the second therapy can comprise a nonsteroidal anti-inflammatory drug and the nonsteroidal anti-inflammatory drug can comprise naproxen, ibuprofen, a salt of any of these, or any combination thereof. In some instances, a NSAID can comprise aspirin, diflunisal, dexibuprofen, oxaprozin, fenoprofen, indomethacin, tolmetin, celecoxib, clonixin, ketoprofen, salts thereof, or any combination thereof. In some instances, a COX-2 inhibitor can comprise etoricoxib, celecoxib, rofecoxib, valdecoxib, a salt thereof, or any combination thereof. In some cases, an active ingredient (e.g. CBD oil) can be combined with another cannabinoid oil. In some embodiments, a composition can comprise an excipient a diluent, a carrier, or any combination thereof.

In some embodiments, a cannabinoid such as CBD can be administered with deoxycholic acid or a salt thereof. In some instances, deoxycholic acid or a salt thereof can increase bioavailability of a cannabinoid or a salt thereof. In some instances, a cannabinoid or a salt thereof can be administered concurrently or consecutively with deoxycholic acid or a salt thereof. In some instances, a cannabinoid or a salt thereof can be formulated into a composition with deoxycholic acid or a salt thereof.

In some embodiments, the composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some embodiments, the composition can be administered so that the active ingredient or the pharmaceutically acceptable salt thereof in the unit dose ranges from about: 500 μg (micrograms) to about 1000 mg, 500 μg to about 700 μg, 600 μg to about 900 μg, 800 μg to about 1 mg (milligram), 1 mg to about 5 mg, 1 mg to about 10 mg, 1 mg to about 200 mg, 2 mg to about 50 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 300 mg to about 600 mg, 400 mg to about 750 mg, 700 mg to about 900 mg, or from about 850 mg to about 1000 mg. In some cases, the unit dose range can be more than about: 500 μg, 550 μg, 600 μg, 650 μg, 700 μg, 750 μg, 800 μg, 850 μg, 900 μg, 950 μg, 1000 μg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg or 100 mg. In some cases, the unit dose range can be less than about: 500 μg, 550 μg, 600 μg, 650 μg, 700 μg, 750 μg, 800 μg, 850 μg, 900 μg, 950 μg, 1000 μg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg or 100 mg. For example, tetrahydrocannabinol (THC), Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, and other cannabinoids can be administered in a unit dose form of about 0.25 mg, about 0.5 mg, about 1.0 mg, about 2.0 mg, about 2.5 mg, about 3.0 mg, about 4.0 mg, about 5.0 mg, about 6.0 mg, about 7.0 mg, about 8.0 mg, about 9.0 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg. Examples of pharmaceutical or dietary supplement composition and methods of administration are shown in Table 3. Different recommended dosing levels are separated by rows by active pharmaceutical ingredient(s).

TABLE 3
Examples of pharmaceutical or dietary supplement compositions

| | | Route of |
|---|---|---|
| Active Pharmaceutical | | Adminis- |
| Ingredient | Recommended Dosing Level | tration |
| CBD (Cannabidiol) | CBD 10 mg | Oral |
| | CBD 25 mg | |
| THC | THC 2.5 mg | Oral |
| (Tetrahydrocannabinol) | THC 5.0 mg | |
| | THC 10.0 mg | |
| THC | THC 2.5 mg, CBD 10 mg | Oral |
| (Tetrahydrocannabinol), | THC 2.5 mg, CBD 25 mg | |
| CBD (Cannabidiol) | THC 5.0 mg, CBD 10 mg | |
| | THC 5.0 mg, CBD 25 mg | |
| | THC 10.0 mg, CBD 10.0 mg | |
| | THC 10.0 mg, CBD 25.0 mg | |
| THCV | THCV 2.5 mg, CBD 10 mg | Oral |
| (Tetrahydrocannabivarin), | THCV 2.5 mg, CBD 25 mg | |
| CBD (Cannabidiol) | THCV 5.0 mg, CBD 10 mg | |
| | THCV 5.0 mg, CBD 25 mg | |
| | THCV 10.0 mg, CBD 10.0 mg | |
| | THCV 10.0 mg, CBD 25.0 mg | |
| Delta-8 (Cannabis- | Delta-8 2.5 mg, CBD 10 mg | Oral |
| or Hemp-derived), | Delta-8 2.5 mg, CBD 25 mg | |
| CBD (Cannabidiol) | Delta-8 5.0 mg, CBD 10 mg | |
| | Delta-8 5.0 mg, CBD 25 mg | |
| | Delta-8 10.0 mg, CBD 10.0 mg | |
| | Delta-8 10.0 mg, CBD 25.0 mg | |
| CBG (Cannabigerol), | CBG 2.5 mg, CBD 10 mg | Oral |
| CBD (Cannabidiol) | CBG 2.5 mg, CBD 25 mg | |
| | CBG 5.0 mg, CBD 10 mg | |
| | CBG 5.0 mg, CBD 25 mg | |
| | CBG 10.0 mg, CBD 10.0 mg | |
| | CNG 10.0 mg, CBD 25.0 mg | |
| Delta-8 (Cannabis- | CBG 2.5 mg, Delta-8 2.5 mg | Oral |
| or Hemp-derived), | CBG 5.0 mg, Delta-8 5.0 mg | |
| CBG (Cannabigerol) | CBG 10.0 mg, Delta-8 10 mg | |
| | CBG 20.0 mg, Delta-8 20 mg | |
| CBN (Cannabinol), | CBN 2.5 mg, CBD 10 mg | Oral |
| CBD (Cannabidiol) | CBN 2.5 mg, CBD 25 mg | |
| | CBN 5.0 mg, CBD 10 mg | |
| | CBN 5.0 mg, CBD 25 mg | |
| | CBN 10.0 mg, CBD 10.0 mg | |
| | CNN 10.0 mg, CBD 25.0 mg | |
| THC (Delta-9) and | 10 mg THC delta-9 and 10 mg | Oral |
| CBD (Full Spectrum) | CBD | |
| | 15 mg THC delta-9 and 10 mg | |
| | CBD | |
| | 20 mg THC delta-9 and 10 mg | |
| | CBD | |
| THC Delta-8 (hemp | 10 mg THC delta-8/10 mg CBD | Oral |
| derived) and CBD | 15 mg THC delta-8/10 mg CBD | |
| (Full Spectrum) | 20 mg THC delta-8/10 mg CBD | |

For Table 3: the exemplary dosing levels for encapsulated pharmaceutical or dietary supplement composition are shown in the columns. When two compounds are shown in a row this indicates both compounds in a composition. For example, the compounds can be blended together, or the compounds can be separated by a capsule in capsule as described herein.

A spray drying manufacturing system for microencapsulated oils can comprise a closed spray drying chamber which can receive a solution comprising a polymer wall material in a suitable solvent mixed with oil droplets comprising the active ingredient (e.g. CBD). Prior to spray drying, a polymer wall material (e.g. hydroxypropyl methylcellulose (HPMC), hydroxypropyl methylcellulose acetate succinate (HPMCAS), cyclodextrins, maltodextrin, or povidone) can be dissolved in a solvent. The active ingredient oil (e.g. CBD) can be made into small oil droplets with a micronizer prior to adding to the dissolved wall material and solvent. The active ingredient (e.g. CBD oil droplets) can be mixed thoroughly with the solvent and wall material to create a homogenous suspension of the oil droplets. The microencapsulated liquid suspension can be fed into the atomizer. In some instances, there can be a spray nozzle at the top of the chamber, where the suspension is atomized with an inert gas, such as air or Nitrogen. When organic solvents are used due to poor water solubility, Nitrogen drying gas can be used to prevent oxidation. The atomizer can be a two component (air/nitrogen and liquid), rotary, hydraulic (“pressure-type”), or ultrasonic nozzle types that distributes the suspension into fine droplets controlled by the atomizer pressure to achieve proper particle size for optimum absorption, release, transfer, or any combination thereof in the gastrointestinal tract. The liquid feed can be converted into small droplets by the atomizer and sprayed into a hot gas path that flash dries the droplets into solid particles. The solvent (aqueous or solvent) can be evaporated and the particles are collected at the exit chamber. The drying chamber can produce uniform fine particles that maintain tight particle size distribution. The particles can be separated from the drying gas using a cyclone separator or filter bag to capture the resulting microencapsulated powder. The spray drying technology can control the particle size and particle size distribution. The process can produce a consistent active ingredient particle size of about 50 microns to about 150 micron range.

With solvent-based spray dry equipment, the process can be a closed loop operation so that the solvents and nitrogen gas may not be exhausted to the atmosphere. The closed-loop system can recycle the nitrogen/solvent outlet gases and pass them through a condenser, reheat and reuse. The closed loop process can recirculate the solvent vapor from the operation back into the system. The amount of solvent condensed out can determine the drying and particle formation. Conversely an open loop cycle could exhaust all gases to the atmosphere in one pass. In some cases, the spray dry equipment can use an open loop cycle, a closed loop cycle or any combination thereof.

After spray drying the particle size can be validated by a particle analyzer prior to blending with an excipient carrier. In some cases, the active powder can be blended with an excipient carrier in a Patterson Kelly (PK Blender) and the blended powder can be fed to a hopper. From the hopper, the dry powder can be placed into a hypromellose capsule, by an encapsulator machine. In some cases, the active powder can be blended with a liquid excipient carrier in a blender and the blended solution can be fed to a hopper. In some cases, the excipient and active ingredient solution can be added to a vegetarian capsule.

### SPECIFIC EMBODIMENTS

A number of compositions, and methods are disclosed herein. Specific exemplary embodiments of these compositions and methods are disclosed below. In some embodiments, compositions and methods herein can be intended for administration to a non-human animal. In some embodiments, compositions and methods herein can be intended for administration to a human.

### Specific Embodiments Section 1

Embodiment 1. A pharmaceutical composition, a dietary supplement composition, or a composition for a non-human animal, in unit dose form, comprising:
i) particles comprising a first active ingredient, or a pharmaceutically acceptable salt thereof wherein the particles are at least partially encapsulated by a coating material and wherein the particles at least partially encapsulated by the coating material are spray dried;
wherein

the particles from i) are at least partially surrounded by a first capsule, a second capsule, or both; and wherein the first capsule is surrounded by the second capsule.

Embodiment 2. The pharmaceutical composition, the dietary supplement composition, or the composition for the non-human animal of embodiment 1, wherein the pharmaceutical composition, the dietary supplement composition, or the composition for a non-human animal further comprises a second active ingredient, or a pharmaceutically acceptable salt thereof in unit dose form.

Embodiment 3. The pharmaceutical composition, the dietary supplement composition, or the composition for the non-human animal of embodiment 1 or 2, wherein the pharmaceutical composition, the dietary supplement composition, or the composition for the non-human animal further comprises an excipient.

Embodiment 4. The pharmaceutical composition, the dietary supplement composition, or the composition for the non-human animal of embodiment 2 or 3, wherein the second active ingredient is at least partially surrounded by the first capsule, the second capsule or both.

Embodiment 5. The pharmaceutical composition, the dietary supplement composition, or the composition for the non-human animal of any one of embodiments 2-4, wherein the second active ingredient is in the form of a liquid, a solid, or a powder in unit dose form.

Embodiment 6. The pharmaceutical composition, the dietary supplement composition, or the composition for the non-human animal of embodiment 5, wherein the second active ingredient is in the form of a liquid in unit dose form, wherein the liquid is an oil.

Embodiment 7. The pharmaceutical composition, the dietary supplement composition, or the composition for the non-human animal of embodiment 2-6, wherein the second active ingredient comprises particles, and wherein the particles are at least partially encapsulated by the coating material and wherein the particles at least partially encapsulated by the coating material are spray dried.

Embodiment 8. The pharmaceutical composition, the dietary supplement composition, or the composition for the non-human animal of embodiment 1 or 7, wherein at least a portion of the particles at least partially encapsulated by the coating material have a particle diameter ranging from about 50 micrometers to about 150 micrometers, as measured by a particle analyzer using laser diffraction.

Embodiment 9. The pharmaceutical composition, the dietary supplement composition, or the composition for the non-human animal of any one of embodiments 2-8, wherein the first active ingredient, the pharmaceutically acceptable salt thereof, the second active ingredient, the pharmaceutically acceptable salt thereof or any combination thereof treats or prevents a disease or a condition or is useful for maintaining health.

Embodiment 10. The pharmaceutical composition, the dietary supplement composition, or the composition for the non-human animal of any one of embodiments 1-9, wherein the first capsule, the second capsule, or both comprise a capsule coating.

Embodiment 11. The pharmaceutical composition, the dietary supplement composition, or the composition for the non-human animal of embodiment 10, wherein the capsule coating at least partially controls active ingredient release.

Embodiment 12. The pharmaceutical composition, the dietary supplement composition, or the composition for the non-human animal of embodiment 10 or 11, wherein the capsule coating comprises an enteric coating, a time release coating, a pH dependent coating, a delayed release coating, an extended release coating, or a combination thereof.

Embodiment 13. The pharmaceutical composition, the dietary supplement composition, or the composition for the non-human animal of any one of embodiments 1-12, wherein the first capsule and the second capsule are formulated to deliver their contents at different locations in the gastrointestinal system.

Embodiment 14. The pharmaceutical composition, the dietary supplement composition, or the composition for the non-human animal of any one of embodiments 1-12, wherein the first capsule and the second capsule are formulated to deliver their contents at about the same location in the gastrointestinal system.

Embodiment 15. The pharmaceutical composition, the dietary supplement composition, or the composition for the non-human animal of any one of embodiments 1-14, wherein the particles at least partially encapsulated by the coating material comprises dissolving the particles comprising the active ingredient or the pharmaceutically acceptable salt thereof in a solvent with the coating material.

Embodiment 16. The pharmaceutical composition, the dietary supplement composition, or the composition for the non-human animal of any one of embodiments 1-15, wherein the coating material comprises a hydroxypropyl methylcellulose (“HPMC”), a hydroxypropyl methylcellulose acetate succinate (“HPMCAS”), a cyclodextrin, a maltodextrin, a povidone, or any combination thereof.

Embodiment 17. The pharmaceutical composition, the dietary supplement composition, or the composition for the non-human animal of any one of embodiments 1-16, wherein the spray drying process comprises: atomizing liquid droplets comprising the active ingredient or the pharmaceutically acceptable salt thereof and the coating material, drying the droplets to form particles, and recovering the particles.

Embodiment 18. The pharmaceutical composition, the dietary supplement composition, or the composition for the non-human animal of embodiment 17, wherein the spray drying process comprises one or more active ingredients.

Embodiment 19. The pharmaceutical composition, the dietary supplement composition, or the composition for the non-human animal of any one of embodiments 2-18, wherein the first active ingredient, the pharmaceutically acceptable salt thereof, the second active ingredient, the pharmaceutically acceptable salt thereof, or any combination thereof is at least partially embedded in the first capsule, the second capsule or both.

Embodiment 20. The pharmaceutical composition, the dietary supplement composition, or the composition for the non-human animal of any one of embodiments 3-19, wherein at least a portion of the excipient and the particles of i) are admixed in a substantially homogenous mixture in unit dose form.

Embodiment 21. The pharmaceutical composition, the dietary supplement composition, or the composition for the non-human animal of any one of embodiments 3-19, wherein at least a portion of the excipient and the second active ingredient, or a pharmaceutically acceptable salt thereof are admixed in a substantially homogenous mixture in unit dose form.

Embodiment 22. The pharmaceutical composition, the dietary supplement composition, or the composition for the non-human animal of any one of embodiments 1-21, further comprising adding a capsule band to the first capsule, the second capsule, or both wherein the capsule band at least partially seals the capsule.

Embodiment 23. The pharmaceutical composition, the dietary supplement composition, or the composition for the non-human animal of any one of embodiments 2-22, wherein the portion of the first capsule not containing the particles comprising the first active ingredient, the pharmaceutically acceptable salt thereof, the second active ingredient, the pharmaceutically acceptable salt thereof, or any combination thereof is at least partially filled with an inert gas.

Embodiment 24. The pharmaceutical composition, the dietary supplement composition, or the composition for the non-human animal of any one of embodiments 1-23, wherein a space exists between the first capsule and the second capsule, and wherein the space is at least in part filled with an inert gas.

Embodiment 25. The pharmaceutical composition, the dietary supplement composition, or the composition for the non-human animal of embodiment 23 or 24, wherein the inert gas comprises nitrogen, carbon dioxide, helium, or any combination thereof.

Embodiment 26. The pharmaceutical composition, the dietary supplement composition, or the composition for the non-human animal of any one of embodiments 3-25, wherein a weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 1000:1 (w/w).

Embodiment 27. The pharmaceutical composition, the dietary supplement composition, or the composition for the non-human animal of embodiment 26, wherein the weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 10:1 (w/w).

Embodiment 28. The pharmaceutical composition, the dietary supplement composition, or the composition for the non-human animal of any one of embodiments 23-27, wherein a content of the first capsule, the second capsule, or both comprise less than about 10% water by weight or wherein a total content of all gases in the capsule is less than about 10% water by weight.

Embodiment 29. The pharmaceutical composition, the dietary supplement composition, or the composition for the non-human animal of any one of embodiments 1-28, wherein the first capsule, the second capsule, or both comprise a hydroxypropyl methylcellulose (HPMC).

Embodiment 30. The pharmaceutical composition, the dietary supplement composition, or the composition for the non-human animal of any one of embodiments 1-29, wherein the first capsule is size: 000, 00, 0, 1, 2, 3, 4, or 5.

Embodiment 31. The pharmaceutical composition, the dietary supplement composition, or the composition for the non-human animal of embodiment 30, wherein the first capsule is size: 1, 2, 3, 4, or 5.

Embodiment 32. The pharmaceutical composition, the dietary supplement composition, or the composition for the non-human animal of any one of embodiments 1-29, wherein the second capsule is size: 000, 00, 0, 1, 2, 3, or 4.

Embodiment 33. The pharmaceutical composition, the dietary supplement composition, or the composition for the non-human animal of embodiment 32, wherein the second capsule is size: 000, 00, 0, or 1.

Embodiment 34. The pharmaceutical composition, the dietary supplement composition, or the composition for the non-human animal of any one of embodiments 1-33, wherein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

Embodiment 35. The pharmaceutical composition, the dietary supplement composition, or the composition for the non-human animal of any one of embodiments 2-34, wherein in a veterinary clinical trial, or an animal safety trial, when ingested, operates mechanistically such that in at least a portion of the animals in the veterinary clinical trial or the animal trial, at least a portion of the second active ingredient is released earlier in time than the particles comprising the first active ingredient.

Embodiment 36. The pharmaceutical composition, the dietary supplement composition, or the composition for the non-human animal of any one of embodiments 3-35, wherein the excipient comprises honey.

Embodiment 37. The pharmaceutical composition, the dietary supplement composition, or the composition for the non-human animal of any one of embodiments 3-35, wherein the excipient comprises honey, glycerin, an oil, a long chain sugar, a polyethylene glycol, a corn syrup, a maltodextrin syrup, a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

Embodiment 38. The pharmaceutical composition, the dietary supplement composition, or the composition for the non-human animal of embodiment 37, wherein the excipient comprises an oil, wherein the oil comprises a food oil.

Embodiment 39. The pharmaceutical composition, the dietary supplement composition, or the composition for the non-human animal of embodiment 38, wherein the food oil comprises an almond oil, an avocado oil, a brazil nut oil, a canola oil, a cashew oil, a chia seed oil, a cocoa butter oil, a coconut oil, a corn oil, a cottonseed oil, a flaxseed/linseed oil, a grape seed oil, a hemp seed oil, a vigna mungo oil, a mustard oil, an olive oil, a bean oil, a palm oil, a peanut oil, a pecan oil, a perilla oil, a rice bran oil, a safflower oil, a sesame oil, a soybean oil, a soybean oil, a walnut oil, a sunflower oil, a cottonseed oil, a vegetable oil or any combination thereof.

Embodiment 40. The pharmaceutical composition, the dietary supplement composition, or the composition for the non-human animal of embodiment 37, wherein the excipient comprises a carbohydrate, wherein the carbohydrate comprises lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

Embodiment 41. The pharmaceutical composition, the dietary supplement composition, or the composition for the non-human animal of any one of embodiments 1-40, wherein the first active ingredient is in the form of a liquid in unit dose form.

Embodiment 42. The pharmaceutical composition, the dietary supplement composition, or the composition for the non-human animal of embodiment 41, wherein the liquid is an oil.

Embodiment 43. The pharmaceutical composition, the dietary supplement composition, or the composition for the non-human animal of any one of embodiments 1-42, wherein the first active ingredient or the pharmaceutically acceptable salt thereof comprises a cannabinoid or a pharmaceutically acceptable salt thereof in unit dose form.

Embodiment 44. The pharmaceutical composition, the dietary supplement composition, or the composition for the non-human animal of any one of embodiments 2-43 wherein the second active ingredient or the pharmaceutically acceptable salt thereof comprises a cannabinoid or a pharmaceutically acceptable salt thereof in unit dose form.

Embodiment 45. The pharmaceutical composition, the dietary supplement composition, or the composition for the non-human animal of embodiment 43 or 44, wherein the cannabinoid comprises tetrahydrocannabinol (THC), tetrahydrocannabinol Delta-8, tetrahydrocannabinol Delta-9, tetrahydrocannabinol Delta-11, tetrahydrocannabinol Delta-13, tetrahydrocannabivarin (THCV), tetrahydrocannabinol THCA, full spectrum THC, or a pharmaceutically acceptable salt thereof in unit dose form.

Embodiment 46. The pharmaceutical composition, the dietary supplement composition, or the composition for the non-human animal of embodiment 43 or 44, wherein the cannabinoid comprises Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, or a pharmaceutically acceptable salt thereof in unit dose form.

Embodiment 47. The pharmaceutical composition, the dietary supplement composition, or the composition for the non-human animal of embodiment 46, wherein the Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, or a pharmaceutically acceptable salt thereof in unit dose form is derived from hemp.

Embodiment 48. The pharmaceutical composition, the dietary supplement composition, or the composition for the non-human animal of any one of embodiments 1-47, wherein the first active ingredient or the pharmaceutically acceptable salt thereof in unit dose form is present in an amount ranging from about 1 mg to about 200 mg, or from about 2 mg to about 50 mg.

Embodiment 49. The pharmaceutical composition, the dietary supplement composition, or the composition for the non-human animal of any one of embodiments 2-48, wherein the second active ingredient or the pharmaceutically acceptable salt thereof in unit dose form is present in an amount ranging from about 1 mg to about 200 mg, or from about 2 mg to about 50 mg

Embodiment 50. The pharmaceutical composition, the dietary supplement composition, or the composition for the non-human animal of any one of embodiments 1-49, further comprising a further active ingredient or a pharmaceutically acceptable salt thereof in unit dose form.

Embodiment 51. The pharmaceutical composition, the dietary supplement composition, or the composition for the non-human animal of embodiment 50, wherein the further active ingredient comprises a cannabinoid, pharmaceutically acceptable salt thereof, or both in unit dose form.

Embodiment 52. The pharmaceutical composition, the dietary supplement composition, or the composition for the non-human animal of embodiment 51, wherein the further active ingredient comprises the cannabinoid, wherein the cannabinoid comprises Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, tetrahydrocannabinol (THC), tetrahydrocannabinol Delta-8, tetrahydrocannabinol Delta-9, tetrahydrocannabinol Delta-11, tetrahydrocannabinol Delta-13, tetrahydrocannabivarin (THCV), tetrahydrocannabinol THCA, full spectrum THC, a pharmaceutically acceptable salt of any of these, or any combination thereof in unit dose form.

Embodiment 53. The pharmaceutical composition, the dietary supplement composition, or the composition for the non-human animal of any one of embodiments 1-52, comprising the salt of the active ingredient, wherein the salt comprises an organic salt, an inorganic salt, or any combination thereof.

Embodiment 54. The pharmaceutical composition, the dietary supplement composition, or the composition for the non-human animal of any one of embodiments 1-52, comprising the salt of the active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof.

Embodiment 55. The pharmaceutical composition, the dietary supplement composition, or the composition for the non-human animal of any one of embodiments 3-54, wherein the excipient is a pharmaceutically acceptable excipient.

Embodiment 56. The pharmaceutical composition, the dietary supplement composition, or the composition for the non-human animal of any one of embodiments 3-54, wherein the excipient is a dietary supplement acceptable excipient.

Embodiment 57. The pharmaceutical composition, the dietary supplement composition, or the composition for the non-human animal of any one of embodiments 1-55, which is a pharmaceutical composition.

Embodiment 58. The pharmaceutical composition of embodiment 57, wherein the first active ingredient or the pharmaceutically acceptable salt thereof comprise an antibiotic, an antiviral, an antiparasitic, a diuretic, a blood pressure medication, a beta-blocker, an ACE inhibitor, an angiotensin II receptor blocker, a calcium channel blocker, an alpha blocker, a cancer chemotherapeutic, a steroid, a nonsteroidal anti-inflammatory drugs (NSAID), an immunomodulator, a pharmaceutically acceptable salt of any of these, or any combination thereof in unit dose form.

Embodiment 59. The pharmaceutical composition of any one of embodiments 57 or 58, wherein the pharmaceutical composition is in a therapeutically effective amount to treat a disease or condition.

Embodiment 60. The pharmaceutical composition, the dietary supplement composition, or the composition for the non-human animal of any one of embodiments 1-54 or 56, which is a composition for a non-human animal.

Embodiment 61. The composition for the non-human animal of embodiment 60, wherein composition for the non-human animal is in effective amount to maintain health of a subject.

Embodiment 62. A kit comprising the pharmaceutical composition, the dietary supplement composition, or the composition for the non-human animal of any one of embodiments 1-61 contained at least in part in a packaging.

Embodiment 63. A method of making a kit, comprising at least partially packaging the pharmaceutical composition, the dietary supplement composition, or the composition for the non-human animal of any one of embodiments 1-61 into a packaging.

Embodiment 64. A method of treating or preventing a disease or condition in a subject in need thereof, comprising treating or preventing the disease or condition by administering a therapeutically effective amount of the pharmaceutical composition of any one of embodiments 57-59 to the subject in need thereof.

Embodiment 65. The method of embodiment 64, wherein the administering is conducted one, two, three, or four times per day.

Embodiment 66. The method of any one of embodiments 64-65, wherein the disease or condition is selected from the group consisting of: cancer, anxiety, pruritus (itching), cognitive function, a chronic pain, pain management, a side effect of chemotherapy, a neurodegenerative disorder, a behavior disorder, sleep aide, appetite stimulant, a seizure, nausea associated with chemotherapy, spinal cord injury, sleep disorders, irritable bowel syndrome, dependency withdrawal, psychiatric symptoms, autoimmune diseases, inflammation, and any combination thereof.

Embodiment 67. The method of any one of embodiments 64-66, wherein the pharmaceutical composition is administered as needed, or for: one day, two days, three days, four days, five days, six days, a week, two weeks, three weeks, a month, two months, three months, four months, five months, six months, seven months, eight months, nine months, ten months, eleven months, a year, or chronically.

Embodiment 68. The method of any one of embodiments 64-67, wherein an amount of the active ingredient or the pharmaceutically acceptable salt thereof in the unit dose ranges from about 1 mg to about 200 mg, or from about 2 mg to about 50 mg

Embodiment 69. The method of any one of embodiments 64-68, wherein a second therapeutic or a pharmaceutically acceptable salt thereof is administered.

Embodiment 70. The method of embodiment 69, wherein the second therapeutic or a pharmaceutically acceptable salt thereof is administered concurrently.

Embodiment 71. The method of embodiment 70, wherein the second therapeutic or the pharmaceutically acceptable salt thereof is comprised in the pharmaceutical formulation.

Embodiment 72. The method of embodiment 71, wherein the wherein the second therapeutic or the pharmaceutically acceptable salt thereof is not comprised in the pharmaceutical formulation.

Embodiment 73. The method of embodiment 69, wherein the second therapeutic or the pharmaceutically acceptable salt thereof is administered consecutively.

Embodiment 74. The method of any one of embodiments 64-73, wherein the subject is diagnosed with the disease or condition.

Embodiment 75. The method of embodiment 74, wherein the diagnosing comprises employing an in vitro diagnostic.

Embodiment 76. The method of embodiment 75, wherein the in vitro diagnostic is a companion diagnostic.

Embodiment 77. The method of any one of embodiments 64-76, wherein the administering is oral administration, rectal administration, or any combination thereof.

Embodiment 78. The method of embodiment 64, wherein the subject is a non-human animal.

Embodiment 79. The method of embodiment 78, wherein the non-human animal is a cat, a dog, a horse, a rodent, a cow, a pig, a bird, a goat, a sheep, or a reptile.

Embodiment 80. The method of embodiment 78, wherein the non-human animal is a dog.

Embodiment 81. The method of embodiment 78, wherein the non-human animal is a cat.

Embodiment 82. The method of embodiment 77, wherein the administering is oral administration and wherein the oral administration is administering the pharmaceutical composition in a food.

Embodiment 83. The method of any one of embodiments 80-82, wherein the subject is over 6 years of age.

Embodiment 84. The method of any one of embodiments 80-82, wherein the subject is under 6 years of age.

Embodiment 85. The method of any one of embodiments 70 or 73, wherein the second therapeutic or the pharmaceutically acceptable salt thereof is administered orally, intranasally, intraocularly, anally, by injection, intravenously, intramuscularly, subcutaneously, intraperitoneally, trans dermally, or any combination thereof.

Embodiment 86. A method of making a pharmaceutical composition, a dietary supplement composition, or a composition for the non-human animal of any one of embodiments 1-61, wherein the method comprises formulating a capsule-in-capsule formulation in unit dose form wherein the formulation comprises:
i) particles comprising the first active ingredient, or a pharmaceutically acceptable salt thereof wherein the particles are at least partially encapsulated by a coating material and wherein the particles at least partially encapsulated by the coating material are spray dried;
wherein

the particles from i) are at least partially surrounded by a first capsule, a second capsule, or both; and wherein the first capsule is surrounded by the second capsule.

Embodiment 87. The method of embodiment 86, wherein the pharmaceutical composition, the dietary supplement composition, or the composition for the non-human animal further comprises a second active ingredient, or a pharmaceutically acceptable salt thereof in unit dose form.

Embodiment 88. The method of embodiment 86 or 87, wherein the pharmaceutical composition, the dietary supplement composition, or the composition for the non-human animal further comprises an excipient.

Embodiment 89. The method of embodiment 87 or 88, wherein the second active ingredient is at least partially surrounded by the first capsule, the second capsule or both.

Embodiment 90. The method of any one of embodiments 87-89, wherein the second active ingredient in unit dose form is in the form of a liquid, a solid, or a powder.

Embodiment 91. The method of any one of embodiments 87-90, wherein the second active ingredient comprises particles, and wherein the particles are at least partially encapsulated by a coating material and wherein the particles at least partially encapsulated by the coating material are spray dried.

Embodiment 92. The method of embodiment 86 or 91, wherein at least a portion of the particles at least partially encapsulated by the coating material have a particle diameter ranging from about 50 micrometers to about 150 micrometers, as measured by a particle analyzer using laser diffraction.

Embodiment 93. The method of any one of embodiments 86-92, wherein the first capsule, the second capsule, or both comprise a capsule coating.

Embodiment 94. The method of embodiment 93, wherein the capsule coating at least partially controls active ingredient release.

Embodiment 95. The method of embodiment 93 or 94, wherein the capsule coating comprises an enteric coating, a time release coating, a pH dependent coating, a delayed release coating, an extended release coating, or a combination thereof.

Embodiment 96. The method of any one of embodiments 86-95, wherein the at least partially encapsulated by the coating material comprises dissolving the particles comprising the active ingredient or the pharmaceutically acceptable salt thereof in a solvent with the coating material.

Embodiment 97. The method of any one of embodiments 86-96, wherein the coating material comprises a hydroxypropyl methylcellulose (“HPMC”), a hydroxypropyl methylcellulose acetate succinate (“HPMCAS”), a cyclodextrin, a maltodextrin, a povidone, or any combination thereof.

Embodiment 98. The method of any one of embodiments 86-97, wherein the spray drying process comprises: atomizing liquid droplets comprising the active ingredient or the pharmaceutically acceptable salt thereof and the coating material, drying the droplets to form particles, and recovering the particles.

Embodiment 99. The method of embodiment 98, wherein the spray drying process comprises one or more active ingredients.

Embodiment 100. The method of any one of embodiments 86-99, further comprising adding a capsule band to the first capsule, the second capsule, or both, wherein the capsule band seals the capsule.

Embodiment 101. The method of any one of embodiments 87-99, wherein the portion of the first capsule not containing the particles comprising the first active ingredient, the pharmaceutically acceptable salt thereof, the second active ingredient, the pharmaceutically acceptable salt thereof, or any combination thereof is at least partially filled with an inert gas.

Embodiment 102. The method of any one of embodiments 86-101, wherein a space exists between the first capsule and the second capsule, and wherein the space is at least in part filled with an inert gas.

Embodiment 103. The method of embodiment 101 or 102, wherein the inert gas comprises nitrogen, carbon dioxide, helium, or any combination thereof.

Embodiment 104. The method of any one of embodiments 86-103, wherein the first capsule, the second capsule, or both are containers that comprise the pharmaceutical composition, the dietary supplement composition, or the composition for the non-human animal.

Embodiment 105. A method of making a pharmaceutical composition, a dietary supplement composition, or a composition for the non-human animal, wherein the method comprises formulating a capsule-in-capsule formulation wherein the formulation comprises:
i) particles comprising a first active ingredient, or a pharmaceutically acceptable salt thereof wherein the particles are at least partially encapsulated by a coating material and wherein the particles at least partially encapsulated by the coating material are spray dried;
wherein

the particles from i) are at least partially surrounded by a first capsule, a second capsule, or both; and wherein the first capsule is surrounded by the second capsule; and wherein the first capsule, the second capsule, or both comprise a capsule coating to at least partially control active ingredient release.

### Specific Embodiments Section 2

Embodiment 1. A pharmaceutical or dietary supplement composition, in unit dose form, comprising:

i) particles comprising a first active ingredient, or a pharmaceutically acceptable salt thereof wherein the particles are at least partially encapsulated by a coating material and wherein the particles at least partially encapsulated by the coating material are spray dried;
wherein
the particles from i) are at least partially surrounded by a first capsule, a second capsule, or both; and wherein the first capsule is surrounded by the second capsule.

Embodiment 2. The pharmaceutical or dietary supplement composition of embodiment 1, wherein the pharmaceutical or dietary supplement composition further comprises a second active ingredient, or a pharmaceutically acceptable salt thereof in unit dose form.

Embodiment 3. The pharmaceutical or dietary supplement composition of embodiment 1 or 2, wherein the pharmaceutical or dietary supplement composition further comprises an excipient.

Embodiment 4. The pharmaceutical or dietary supplement composition of embodiment 2 or 3, wherein the second active ingredient is at least partially surrounded by the first capsule, the second capsule or both.

Embodiment 5. The pharmaceutical or dietary supplement composition of any one of embodiments 2-4, wherein the second active ingredient is in the form of a liquid, a solid, or a powder in unit dose form.

Embodiment 6. The pharmaceutical or dietary supplement composition of embodiment 5, wherein the second active ingredient is in the form of a liquid in unit dose form, wherein the liquid is an oil.

Embodiment 7. The pharmaceutical or dietary supplement composition of embodiment 2-6, wherein the second active ingredient comprises particles, and wherein the particles are at least partially encapsulated by the coating material and wherein the particles at least partially encapsulated by the coating material are spray dried.

Embodiment 8. The pharmaceutical or dietary supplement composition of embodiment 1 or 7, wherein at least a portion of the particles at least partially encapsulated by the coating material have a particle diameter ranging from about 50 micrometers to about 150 micrometers, as measured by a particle analyzer using laser diffraction.

Embodiment 9. The pharmaceutical or dietary supplement composition of any one of embodiments 2-8, wherein the first active ingredient, the pharmaceutically acceptable salt thereof, the second active ingredient, the pharmaceutically acceptable salt thereof or any combination thereof treats or prevents a disease or a condition or is useful for maintaining health.

Embodiment 10. The pharmaceutical or dietary supplement composition of any one of embodiments 1-9, wherein the first capsule, the second capsule, or both comprise a capsule coating.

Embodiment 11. The pharmaceutical or dietary supplement composition of embodiment 10, wherein the capsule coating at least partially controls active ingredient release.

Embodiment 12. The pharmaceutical or dietary supplement composition of embodiment 10 or 11, wherein the capsule coating comprises an enteric coating, a time release coating, a pH dependent coating, a delayed release coating, an extended release coating, or a combination thereof.

Embodiment 13. The pharmaceutical or dietary supplement composition of any one of embodiments 1-12, wherein the first capsule and the second capsule are formulated to deliver their contents at different locations in the gastrointestinal system.

Embodiment 14. The pharmaceutical or dietary supplement composition of any one of embodiments 1-12, wherein the first capsule and the second capsule are formulated to deliver their contents at about the same location in the gastrointestinal system.

Embodiment 15. The pharmaceutical or dietary supplement composition of any one of embodiments 1-14, wherein the particles at least partially encapsulated by the coating material comprises dissolving the particles comprising the active ingredient or the pharmaceutically acceptable salt thereof in a solvent with the coating material.

Embodiment 16. The pharmaceutical or dietary supplement composition of any one of embodiments 1-15, wherein the coating material comprises a hydroxypropyl methylcellulose (“HPMC”), a hydroxypropyl methylcellulose acetate succinate (“HPMCAS”), a cyclodextrin, a maltodextrin, a povidone, or any combination thereof.

Embodiment 17. The pharmaceutical or dietary supplement composition of any one of embodiments 1-16, wherein the spray drying process comprises: atomizing liquid droplets comprising the active ingredient or the pharmaceutically acceptable salt thereof and the coating material, drying the droplets to form particles, and recovering the particles.

Embodiment 18. The pharmaceutical or dietary supplement composition of embodiment 17, wherein the spray drying process comprises one or more active ingredients.

Embodiment 19. The pharmaceutical or dietary supplement composition of any one of embodiments 2-18, wherein the first active ingredient, the pharmaceutically acceptable salt thereof, the second active ingredient, the pharmaceutically acceptable salt thereof, or any combination thereof is at least partially embedded in the first capsule, the second capsule or both.

Embodiment 20. The pharmaceutical or dietary supplement composition of any one of embodiments 3-19, wherein at least a portion of the excipient and the particles of i) are admixed in a substantially homogenous mixture in unit dose form.

Embodiment 21. The pharmaceutical or dietary supplement composition of any one of embodiments 3-19, wherein at least a portion of the excipient and the second active ingredient, or a pharmaceutically acceptable salt thereof are admixed in a substantially homogenous mixture in unit dose form.

Embodiment 22. The pharmaceutical or dietary supplement composition of any one of embodiments 1-21, further comprising adding a capsule band to the first capsule, the second capsule, or both wherein the capsule band at least partially seals the capsule.

Embodiment 23. The pharmaceutical or dietary supplement composition of any one of embodiments 2-22, wherein the portion of the first capsule not containing the particles comprising the first active ingredient, the pharmaceutically acceptable salt thereof, the second active ingredient, the pharmaceutically acceptable salt thereof, or any combination thereof is at least partially filled with an inert gas.

Embodiment 24. The pharmaceutical or dietary supplement composition of any one of embodiments 1-23, wherein a space exists between the first capsule and the second capsule, and wherein the space is at least in part filled with an inert gas.

Embodiment 25. The pharmaceutical or dietary supplement composition of embodiment 23 or 24, wherein the inert gas comprises nitrogen, carbon dioxide, helium, or any combination thereof.

Embodiment 26. The pharmaceutical or dietary supplement composition of any one of embodiments 3-25, wherein a weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 1000:1 (w/w).

Embodiment 27. The pharmaceutical or dietary supplement composition of embodiment 26, wherein the weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 10:1 (w/w).

Embodiment 28. The pharmaceutical or dietary supplement composition of any one of embodiments 23-27, wherein a content of the first capsule, the second capsule, or both comprise less than about 10% water by weight or wherein a total content of all gases in the capsule is less than about 10% water by weight.

Embodiment 29. The pharmaceutical or dietary supplement composition of any one of embodiments 1-28, wherein the first capsule, the second capsule, or both comprise a hydroxypropyl methylcellulose (HPMC).

Embodiment 30. The pharmaceutical or dietary supplement composition of any one of embodiments 1-29, wherein the first capsule is size: 000, 00, 0, 1, 2, 3, 4, or 5.

Embodiment 31. The pharmaceutical or dietary supplement composition of embodiment 30, wherein the first capsule is size: 1, 2, 3, 4, or 5.

Embodiment 32. The pharmaceutical or dietary supplement composition of any one of embodiments 1-29, wherein the second capsule is size: 000, 00, 0, 1, 2, 3, or 4.

Embodiment 33. The pharmaceutical or dietary supplement composition of embodiment 32, wherein the second capsule is size: 000, 00, 0, or 1.

Embodiment 34. The pharmaceutical or dietary supplement composition of any one of embodiments 1-33, wherein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

Embodiment 35. The pharmaceutical or dietary supplement composition of any one of embodiments 2-34, wherein in a human clinical trial or human safety trial, when ingested, operates mechanistically such that in at least a portion of the humans in the clinical trial or the safety trial, at least a portion of the second active ingredient is released earlier in time than the particles comprising the first active ingredient.

Embodiment 36. The pharmaceutical or dietary supplement composition of any one of embodiments 3-35, wherein the excipient comprises honey.

Embodiment 37. The pharmaceutical or dietary supplement composition of any one of embodiments 3-35, wherein the excipient comprises honey, glycerin, an oil, a long chain sugar, a polyethylene glycol, a corn syrup, a maltodextrin syrup, a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

Embodiment 38. The pharmaceutical or dietary supplement composition of embodiment 37, wherein the excipient comprises an oil, wherein the oil comprises a food oil.

Embodiment 39. The pharmaceutical or dietary supplement composition of embodiment 38, wherein the food oil comprises an almond oil, an avocado oil, a brazil nut oil, a canola oil, a cashew oil, a chia seed oil, a cocoa butter oil, a coconut oil, a corn oil, a cottonseed oil, a flaxseed/linseed oil, a grape seed oil, a hemp seed oil, a vigna mungo oil, a mustard oil, an olive oil, a bean oil, a palm oil, a peanut oil, a pecan oil, a perilla oil, a rice bran oil, a safflower oil, a sesame oil, a soybean oil, a soybean oil, a walnut oil, a sunflower oil, a cottonseed oil, a vegetable oil or any combination thereof.

Embodiment 40. The pharmaceutical or dietary supplement composition of embodiment 37, wherein the excipient comprises a carbohydrate, wherein the carbohydrate comprises lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

Embodiment 41. The pharmaceutical or dietary supplement composition of any one of embodiments 1-40, wherein the first active ingredient is in the form of a liquid in unit dose form.

Embodiment 42. The pharmaceutical or dietary supplement composition of embodiment 41, wherein the liquid is an oil.

Embodiment 43. The pharmaceutical or dietary supplement composition of any one of embodiments 1-42, wherein the first active ingredient or the pharmaceutically acceptable salt thereof comprises a cannabinoid or a pharmaceutically acceptable salt thereof in unit dose form.

Embodiment 44. The pharmaceutical or dietary supplement composition of any one of embodiments 2-43 wherein the second active ingredient or the pharmaceutically acceptable salt thereof comprises a cannabinoid or a pharmaceutically acceptable salt thereof in unit dose form.

Embodiment 45. The pharmaceutical or dietary supplement composition of embodiment 43 or 44, wherein the cannabinoid comprises tetrahydrocannabinol (THC), tetrahydrocannabinol Delta-8, tetrahydrocannabinol Delta-9, tetrahydrocannabinol Delta-11, tetrahydrocannabinol Delta-13, tetrahydrocannabivarin (THCV), tetrahydrocannabinol THCA, full spectrum THC, or a pharmaceutically acceptable salt thereof in unit dose form.

Embodiment 46. The pharmaceutical or dietary supplement composition of embodiment 43 or 44, wherein the cannabinoid comprises Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, or a pharmaceutically acceptable salt thereof in unit dose form.

Embodiment 47. The pharmaceutical or dietary supplement composition of embodiment 46, wherein the Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, or a pharmaceutically acceptable salt thereof in unit dose form is derived from hemp.

Embodiment 48. The pharmaceutical or dietary supplement composition of any one of embodiments 1-47, wherein the first active ingredient or the pharmaceutically acceptable salt thereof in unit dose form is present in an amount ranging from about 1 mg to about 200 mg, or from about 2 mg to about 50 mg.

Embodiment 49. The pharmaceutical or dietary supplement composition of any one of embodiments 2-48, wherein the second active ingredient or the pharmaceutically acceptable salt thereof in unit dose form is present in an amount ranging from about 1 mg to about 200 mg, or from about 2 mg to about 50 mg

Embodiment 50. The pharmaceutical or dietary supplement composition of any one of embodiments 1-49, further comprising a further active ingredient or a pharmaceutically acceptable salt thereof in unit dose form.

Embodiment 51. The pharmaceutical or dietary supplement composition of embodiment 50, wherein the further active ingredient comprises a cannabinoid, pharmaceutically acceptable salt thereof, or both in unit dose form.

Embodiment 52. The pharmaceutical or dietary supplement composition of embodiment 51, wherein the further active ingredient comprises the cannabinoid, wherein the cannabinoid comprises Cannabichromene (CBC), Cannabichromevarin (CBCV), Cannabidiol (CBD), Cannabidivarin (CBDV), Cannabigerol (CBG), Cannabigerivarin (CBGV), Cannabinol (CBN), Cannabivarin (CBV), THC Delta-8, tetrahydrocannabinol (THC), tetrahydrocannabinol Delta-8, tetrahydrocannabinol Delta-9, tetrahydrocannabinol Delta-11, tetrahydrocannabinol Delta-13, tetrahydrocannabivarin (THCV), tetrahydrocannabinol THCA, full spectrum THC, a pharmaceutically acceptable salt of any of these, or any combination thereof in unit dose form.

Embodiment 53. The pharmaceutical or dietary supplement composition of any one of embodiments 1-52, comprising the salt of the active ingredient, wherein the salt comprises an organic salt, an inorganic salt, or any combination thereof.

Embodiment 54. The pharmaceutical or dietary supplement composition of any one of embodiments 1-52, comprising the salt of the active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof.

Embodiment 55. The pharmaceutical or dietary supplement composition of any one of embodiments 3-54, wherein the excipient is a pharmaceutically acceptable excipient.

Embodiment 56. The pharmaceutical or dietary supplement composition of any one of embodiments 3-54, wherein the excipient is a dietary supplement acceptable excipient.

Embodiment 57. The pharmaceutical or dietary supplement composition of any one of embodiments 1-55, which is a pharmaceutical composition.

Embodiment 58. The pharmaceutical composition of embodiment 57, wherein the first active ingredient or the pharmaceutically acceptable salt thereof comprise an antibiotic, an antiviral, an antiparasitic, a diuretic, a blood pressure medication, a beta-blocker, an ACE inhibitor, an angiotensin II receptor blocker, a calcium channel blocker, an alpha blocker, a cancer chemotherapeutic, a steroid, a nonsteroidal anti-inflammatory drugs (NSAID), an immunomodulator, a pharmaceutically acceptable salt of any of these, or any combination thereof in unit dose form.

Embodiment 59. The pharmaceutical composition of any one of embodiments 57 or 58, wherein the pharmaceutical composition is in a therapeutically effective amount to treat a disease or condition.

Embodiment 60. The pharmaceutical or dietary supplement composition of any one of embodiments 1-54 or 56, which is a dietary supplement composition.

Embodiment 61. The dietary supplement composition of embodiment 60, wherein the dietary supplement is in effective amount to maintain health of a subject.

Embodiment 62. A kit comprising the pharmaceutical or dietary supplement composition of any one of embodiments 1-61 contained at least in part in a packaging.

Embodiment 63. A method of making a kit, comprising at least partially packaging the pharmaceutical or dietary supplement composition of any one of embodiments 1-61 into a packaging.

Embodiment 64. A method of treating or preventing a disease or condition in a subject in need thereof, comprising treating or preventing the disease or condition by administering a therapeutically effective amount of the pharmaceutical composition of any one of embodiments 57-59 to the subject in need thereof.

Embodiment 65. The method of embodiment 64, wherein the administering is conducted one, two, three, or four times per day.

Embodiment 66. The method of any one of embodiments 64-65, wherein the disease or condition is selected from the group consisting of: cancer, breast cancer, melanoma, anxiety, pruritus (itching), cognitive function, Alzheimer's disease, amyotrophic lateral sclerosis, Huntington's disease, Parkinson's disease, a chronic pain, pain management, multiple sclerosis, a side effect of chemotherapy, HIV, AIDS, a neurodegenerative disorder, a behavior disorder, Tourette syndrome, cervical dystonia, sleep aide, appetite stimulant, a seizure, an epilepsy, nausea associated with chemotherapy, anorexia, spinal cord injury, sleep disorders, glaucoma, schizophrenia, epilepsy, asthma, posttraumatic stress disorder, cachexia, irritable bowel syndrome, dependency withdrawal, psychiatric symptoms, autoimmune diseases, inflammation, sleep apnea, a headache, a migraine, opioid addiction, and any combination thereof.

Embodiment 67. The method of any one of embodiments 64-66, wherein the pharmaceutical composition is administered as needed, or for: one day, two days, three days, four days, five days, six days, a week, two weeks, three weeks, a month, two months, three months, four months, five months, six months, seven months, eight months, nine months, ten months, eleven months, a year, or chronically.

Embodiment 68. The method of any one of embodiments 64-67, wherein an amount of the active ingredient or the pharmaceutically acceptable salt thereof in the unit dose ranges from about 1 mg to about 200 mg, or from about 2 mg to about 50 mg

Embodiment 69. The method of any one of embodiments 64-68, wherein a second therapeutic or a pharmaceutically acceptable salt thereof is administered.

Embodiment 70. The method of embodiment 69, wherein the second therapeutic or a pharmaceutically acceptable salt thereof is administered concurrently.

Embodiment 71. The method of embodiment 70, wherein the second therapeutic or the pharmaceutically acceptable salt thereof is comprised in the pharmaceutical formulation.

Embodiment 72. The method of embodiment 71, wherein the wherein the second therapeutic or the pharmaceutically acceptable salt thereof is not comprised in the pharmaceutical formulation.

Embodiment 73. The method of embodiment 69, wherein the second therapeutic or the pharmaceutically acceptable salt thereof is administered consecutively.

Embodiment 74. The method of any one of embodiments 64-73, wherein the subject is diagnosed with the disease or condition.

Embodiment 75. The method of embodiment 74, wherein the diagnosing comprises employing an in vitro diagnostic.

Embodiment 76. The method of embodiment 75, wherein the in vitro diagnostic is a companion diagnostic.

Embodiment 77. The method of any one of embodiments 64-76, wherein the administering is oral administration, rectal administration, or any combination thereof.

Embodiment 78. The method of embodiment 64, wherein the subject is a non-human animal.

Embodiment 79. The method of embodiment 78, wherein the non-human animal is a cat, a dog, a horse, a rodent, a cow, a pig, a bird, a goat, a sheep, or a reptile.

Embodiment 80. The method of embodiment 64, wherein the subject is a human.

Embodiment 81. The method of embodiment 80, wherein the subject is a man.

Embodiment 82. The method of embodiment 80, wherein the subject is a woman.

Embodiment 83. The method of any one of embodiments 80-82, wherein the subject is over 18 years of age.

Embodiment 84. The method of any one of embodiments 80-82, wherein the subject is under 18 years of age.

Embodiment 85. The method of any one of embodiments 70 or 73, wherein the second therapeutic or the pharmaceutically acceptable salt thereof is administered orally, intranasally, intraocularly, anally, by injection, intravenously, intramuscularly, subcutaneously, intraperitoneally, trans dermally, or any combination thereof.

Embodiment 86. A method of making the pharmaceutical or dietary supplement composition of any one of embodiments 1-61, wherein the method comprises formulating a capsule-in-capsule formulation in unit dose form wherein the formulation comprises:

i) particles comprising the first active ingredient, or a pharmaceutically acceptable salt thereof wherein the particles are at least partially encapsulated by a coating material and wherein the particles at least partially encapsulated by the coating material are spray dried;
wherein
the particles from i) are at least partially surrounded by a first capsule, a second capsule, or both; and wherein the first capsule is surrounded by the second capsule.

Embodiment 87. The method of embodiment 86, wherein the pharmaceutical or dietary supplement composition further comprises a second active ingredient, or a pharmaceutically acceptable salt thereof in unit dose form.

Embodiment 88. The method of embodiment 86 or 87, wherein the pharmaceutical or dietary supplement composition further comprises an excipient.

Embodiment 89. The method of embodiment 87 or 88, wherein the second active ingredient is at least partially surrounded by the first capsule, the second capsule or both.

Embodiment 90. The method of any one of embodiments 87-89, wherein the second active ingredient in unit dose form is in the form of a liquid, a solid, or a powder.

Embodiment 91. The method of any one of embodiments 87-90, wherein the second active ingredient comprises particles, and wherein the particles are at least partially encapsulated by a coating material and wherein the particles at least partially encapsulated by the coating material are spray dried.

Embodiment 92. The method of embodiment 86 or 91, wherein at least a portion of the particles at least partially encapsulated by the coating material have a particle diameter ranging from about 50 micrometers to about 150 micrometers, as measured by a particle analyzer using laser diffraction.

Embodiment 93. The method of any one of embodiments 86-92, wherein the first capsule, the second capsule, or both comprise a capsule coating.

Embodiment 94. The method of embodiment 93, wherein the capsule coating at least partially controls active ingredient release.

Embodiment 95. The method of embodiment 93 or 94, wherein the capsule coating comprises an enteric coating, a time release coating, a pH dependent coating, a delayed release coating, an extended release coating, or a combination thereof.

Embodiment 96. The method of any one of embodiments 86-95, wherein the at least partially encapsulated by the coating material comprises dissolving the particles comprising the active ingredient or the pharmaceutically acceptable salt thereof in a solvent with the coating material.

Embodiment 97. The method of any one of embodiments 86-96, wherein the coating material comprises a hydroxypropyl methylcellulose (“HPMC”), a hydroxypropyl methylcellulose acetate succinate (“HPMCAS”), a cyclodextrin, a maltodextrin, a povidone, or any combination thereof.

Embodiment 98. The method of any one of embodiments 86-97, wherein the spray drying process comprises: atomizing liquid droplets comprising the active ingredient or the pharmaceutically acceptable salt thereof and the coating material, drying the droplets to form particles, and recovering the particles.

Embodiment 99. The method of embodiment 98, wherein the spray drying process comprises one or more active ingredients.

Embodiment 100. The method of any one of embodiments 86-99, further comprising adding a capsule band to the first capsule, the second capsule, or both, wherein the capsule band seals the capsule.

Embodiment 101. The method of any one of embodiments 87-99, wherein the portion of the first capsule not containing the particles comprising the first active ingredient, the pharmaceutically acceptable salt thereof, the second active ingredient, the pharmaceutically acceptable salt thereof, or any combination thereof is at least partially filled with an inert gas.

Embodiment 102. The method of any one of embodiments 86-101, wherein a space exists between the first capsule and the second capsule, and wherein the space is at least in part filled with an inert gas.

Embodiment 103. The method of embodiment 101 or 102, wherein the inert gas comprises nitrogen, carbon dioxide, helium, or any combination thereof.

Embodiment 104. The method of any one of embodiments 86-103, wherein the first capsule, the second capsule, or both are containers that comprise the pharmaceutical or dietary supplement composition.

Embodiment 105. A method of making a pharmaceutical or dietary supplement composition, wherein the method comprises formulating a capsule-in-capsule formulation wherein the formulation comprises:

i) particles comprising a first active ingredient, or a pharmaceutically acceptable salt thereof wherein the particles are at least partially encapsulated by a coating material and wherein the particles at least partially encapsulated by the coating material are spray dried;
wherein
the particles from i) are at least partially surrounded by a first capsule, a second capsule, or both; and wherein the first capsule is surrounded by the second capsule;
and wherein the first capsule, the second capsule, or both comprise a capsule coating to at least partially control active ingredient release.

### EXAMPLES

The following examples are included for illustrative purposes only and are not intended to limit the scope of the disclosure.

### Example 1

A male subject will be diagnosed with anxiety. The subject will be prescribed a dosing regimen of a pharmaceutical composition. The pharmaceutical composition will comprise encapsulated THC Delta-8 at 2.5 mg and CBD at 25 mg which are processed separately to a dry powder using the methods described herein (e.g. spay drying). The THC Delta-8 dry powder will be mixed with a long chain polysaccharide and encapsulated into a first capsule. The first capsule will be sealed and coated with a pH dependent coating. The CBD dry powder will be mixed with a vegetable oil and placed into the second capsule. Additionally, the first capsule will be placed into the second capsule. The second capsule will be sealed and coated with an enteric coating. The capsule-in-capsule THC Delta-8 and CBD will be administered orally. The absorption of the CBD pharmaceutical composition will reach the blood stream at least 2X faster than the THC Delta-8 pharmaceutical composition.

### Example 2

A subject will be diagnosed with chronic pain. The subject will be prescribed a dosing regimen of a pharmaceutical composition. The pharmaceutical composition will comprise encapsulated THC and CBD which are processed to a dry powder using the methods described herein (e.g. spay drying). The THC and CBD dry powder will be mixed together with a long chain polysaccharide and encapsulated into a first capsule. The first capsule will be sealed and coated with a time-release dependent coating. A second capsule will encapsulate the first capsule and will be sealed. The second capsule will be coated with a time-release dependent coating. The pharmaceutical composition will be administered to the subject by a rectal suppository. The dosing regimen will comprise an effective amount (e.g. 0.5 mg, 1.0 mg, 2.5 mg or 5.0 mg) of the combined THC and CBD encapsulated particles at a 1:1 ratio to treat the disease. A dosing level of the suppository administered THC and CBD pharmaceutical composition will be about 10% lower than a subject receiving the oral administration of THC and CBD.

### Example 3

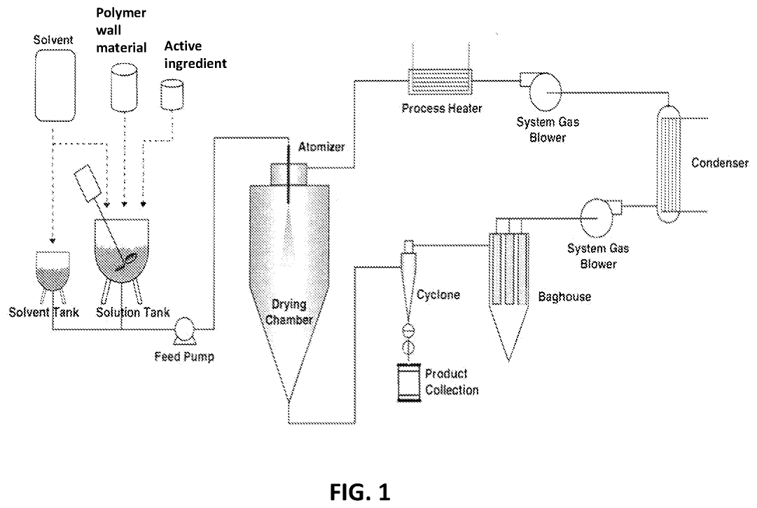

The active encapsulated ingredient (e.g. CBD) in a pharmaceutical composition described herein is manufactured by a spray drying system. FIG. 1 shows a spray drying manufacturing system comprising a closed spray drying chamber which receives the solution comprising a polymer wall material in a suitable solvent mixed with oil droplets comprising the active ingredient (e.g. CBD). Prior to spray drying, a polymer wall material (e.g. hydroxypropyl methylcellulose (HPMC), hydroxypropyl methylcellulose acetate succinate (HPMCAS), cyclodextrins, maltodextrin, or povidone) is dissolved in a solvent. The active ingredient oil (e.g. CBD) can be made into small oil droplets with a micronizer prior to adding to the dissolved wall material and solvent. The active ingredient (e.g. CBD oil droplets) is mixed thoroughly with the solvent and wall material to create a homogenous suspension of the oil droplets. The solution is fed into an atomizer located at the top of the chamber. The atomizer is a two component or rotary nozzle type that distributes the solution into fine droplets controlled by the atomizer pressure. This atomization gas is an inert gas, such as air, nitrogen or carbon dioxide. The atomized droplets go through a drying chamber with hot gas to produce uniform fine particles that maintain a tight particle size distribution following liquid evaporation. Solid particle form and fall to the bottom of the drying chamber. The balance between temperature, flow rate and droplet size control the drying process. The powder is recovered from the exhaust gas using a cyclone or a bag filter. Particle size is validated by a Malvern particle analyzer prior to blending with an excipient carrier.

The active powder is blended with an excipient carrier (honey) in a blender and the blended active ingredient is fed to a hopper. From the hopper, the first active ingredient is loaded into a Size 4 Hypromellose capsule, with a piston pump or a rotary pump.

The capsule halves are then closed together to form an enclosed capsule that contains the active cannabinoid and excipient honey. A capsule band is added to the capsule and the pH-dependent capsule coating is applied to the capsule. This process is repeated with a second encapsulated ingredient (e.g. another cannabinoid). Once the second cannabinoid is mixed with an excipient carrier, the second cannabinoid and excipient are a loaded into a size 00 capsule. Additionally, the size 4 capsule containing the first active ingredient is added to the size 00 capsule containing the second cannabinoid. The 00 capsule is closed, and a capsule band is added. The capsule is packaged in unit dose form.

### Example 4

A subject will be diagnosed with multiple sclerosis. The subject will be prescribed a dosing regimen of a pharmaceutical composition. The pharmaceutical composition will comprise encapsulated THC Delta-8, THC Delta-9, and THC Delta-11 which are processed separately to a dry powder using the methods described herein (e.g. spray drying). The THC Delta-8 dry powder and will be mixed with polyethylene glycol and encapsulated into a first capsule. The first capsule will be sealed and coated with a time-release coating. The THC Delta-9, and THC Delta-11 will be mixed with a medium-chain triglyceride oil and placed into the second capsule. Additionally, the first capsule will be placed into the second capsule. The second capsule will be sealed and coated with a pH dependent coating. The capsule-in-capsule THC Delta-8, THC Delta-9, and THC Delta-11 will be administered orally. The THC Delta-9, and THC Delta-11 pharmaceutical composition will reach the blood stream at least 3X faster than the THC Delta-8 pharmaceutical composition which is configured to be released in the colon.

### Example 5

A 74-year-old female subject with a history of hip pain was administered a capsule in capsule formulation containing 10 mg CBD. One capsule in capsule pill was administered in the evening for a total dose of 10 mg of CBD in a 24-hour period. The female subject's hip pain was reduced in 2-5 days after daily administration of the 10 mg of CBD as compared to her hip pain prior to the administration of CBD. The subject stopped taking the CBD for 1-2 weeks and severe pain returned. The subject resumed 10 mg daily of the capsule in capsule CBD formulation and the pain subsided after administration for several days.

### Example 6

A 64-year-old male subject with rheumatoid arthritis was administered a capsule in capsule formulation containing 10 mg CBD. One capsule in capsule pill was administered in the evening for a total dose of 10 mg of CBD in a 24-hour period. The male subject's arthritis symptoms such as pain and swelling were reduced in 2-5 days after daily administration of the 10 mg of CBD as compared to his arthritis symptoms (e.g., pain and swelling) prior to the administration of CBD. For example, the swelling the subject's hands was reduced.

### Example 7

A male subject in his 20's with sickle cell anemia was administered a capsule in capsule formulation containing 10 mg CBD. Two capsule in capsule pills were administered in the evening for a total dose of 20 mg of CBD in a 24-hour period. The male subject's sickle cell symptoms were reduced in 2-3 days after daily administration of the 20 mg of CBD as compared to his sickle cell symptoms prior to the administration of CBD. For example, the subject was able to get a 6-8 hours of continuous sleep after treatment as compared to waking up multiple times throughout the night prior to treatment.

### Example 8

A female subject with ulcerative colitis was administered a capsule in capsule formulation containing 10 mg CBD. Two capsule in capsule pills were administered in the morning and one capsule in capsule pill was administered in the evening for a total dose of 30 mg of CBD in a 24 hour period. The female subject's ulcerative colitis symptoms such as irritable bowel symptoms were reduced after daily administration of the 30 mg of CBD as compared to her ulcerative colitis symptoms prior to the administration of CBD. For example, the subject had reduced abdominal pain and bloating after treatment as compared to her abdominal pain and bloating before treatment.

### Example 9

A 73-year-old male subject with Parkinson's Disease was administered a capsule in capsule formulation containing 10 mg of microencapsulated CBD engineered to release into the intestine. Two capsule in capsule pills were administered per day for a total dose of 20 mg of CBD in a 24 hour period. The male subject's Parkinson's disease symptoms such as nightmares during sleeping were reduced after daily administration of the 20 mg of CBD as compared to his nightmares prior to the administration of CBD. Additionally, the subject was diagnosed with arthritis and has had decreased pain and swelling in his fingers since the administration of the microencapsulated CBD.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the methods presented in the disclosure. It is intended that the following claims define the scope of the disclosure and that methods and structures within the scope of these claims and their equivalents be covered thereby.

What is claimed is:

1. A pharmaceutical or dietary supplement composition, in unit dose form, comprising:
a first active ingredient or a pharmaceutically acceptable salt thereof comprising
a plurality of spray dried particles, each particle of the plurality of spray dried particles comprising a cannabinoid or a pharmaceutically acceptable salt thereof, substantially encapsulated in a coating material;
wherein within the plurality of spray dried particles comprising the cannabinoid, or the pharmaceutically acceptable salt thereof, substantially encapsulated in the coating material, individually have a particle diameter ranging from about 50 micrometers to about 150 micrometers, as measured by a particle analyzer using laser diffraction;
wherein the coating material comprises a hydroxypropyl methylcellulose (HPMC), a hydroxypropyl methylcellulose acetate succinate (HPMCAS), a maltodextrin, a povidone, a copovidone or any combination thereof; and
wherein the plurality of spray dried particles are at least partially surrounded by a first capsule, a second capsule, or both; and wherein the first capsule is surrounded by the second capsule.

2. The pharmaceutical or dietary supplement composition of claim 1, wherein the pharmaceutical or dietary supplement composition further comprises a second active ingredient, or a pharmaceutically acceptable salt thereof in unit dose form.

3. The pharmaceutical or dietary supplement composition of claim 1 or 2, wherein the pharmaceutical or dietary supplement composition further comprises an excipient.

4. The pharmaceutical or dietary supplement composition of claim 2 or 3, wherein the second active ingredient is at least partially surrounded by the first capsule, the second capsule or both.

5. The pharmaceutical or dietary supplement composition of any one of claims 2-4, wherein the second active ingredient is in the form of a liquid, a solid, or a powder in unit dose form.

6. The pharmaceutical or dietary supplement composition of claim 5, wherein the second active ingredient is in the form of a liquid in unit dose form, wherein the liquid is an oil.

7. The pharmaceutical or dietary supplement composition of claim 2-6, wherein the second active ingredient comprises particles, and wherein the particles are at least partially encapsulated by the coating material and wherein the particles at least partially encapsulated by the coating material are spray dried.

8. The pharmaceutical or dietary supplement composition of claim 7, wherein at least a portion of the particles at least partially encapsulated by the coating material have a particle diameter ranging from about 50 micrometers to about 150 micrometers, as measured by a particle analyzer using laser diffraction.

9. The pharmaceutical or dietary supplement composition of any one of claims 2-8, wherein the first active ingredient, the pharmaceutically acceptable salt thereof, the second active ingredient, the pharmaceutically acceptable salt thereof or any combination thereof treats or prevents a disease or a condition or is useful for maintaining health.

10. The pharmaceutical or dietary supplement composition of any one of claims 1-9, wherein the first capsule, the second capsule, or both comprise a capsule coating.

11. The pharmaceutical or dietary supplement composition of claim 10, wherein the capsule coating at least partially controls active ingredient release.

12. The pharmaceutical or dietary supplement composition of claim 10 or 11, wherein the capsule coating comprises an enteric coating, a time release coating, a pH dependent coating, a delayed release coating, an extended-release coating, or a combination thereof.

13. The pharmaceutical or dietary supplement composition of any one of claims 1-12, wherein the first capsule and the second capsule are formulated to deliver their contents at different locations in the gastrointestinal system.

14. The pharmaceutical or dietary supplement composition of any one of claims 1-12, wherein the first capsule and the second capsule are formulated to deliver their contents at about the same location in the gastrointestinal system.

15. The pharmaceutical or dietary supplement composition of any one of claims 1-14, wherein the particles at least partially encapsulated by the coating material comprises dissolving the particles comprising the active ingredient or the pharmaceutically acceptable salt thereof in a solvent with the coating material.

16. The pharmaceutical or dietary supplement composition of any one of claims 1-15, comprising the coating material, wherein the coating material comprises the hydroxypropyl methylcellulose acetate succinate (“HPMCAS”).

17. The pharmaceutical or dietary supplement composition of any one of claims 1-16, wherein the spray drying process comprises: atomizing liquid droplets comprising the active ingredient or the pharmaceutically acceptable salt thereof and the coating material, drying the droplets to form particles, and recovering the particles.

18. The pharmaceutical or dietary supplement composition of claim 17, wherein the spray drying process comprises one or more active ingredients.

19. The pharmaceutical or dietary supplement composition of any one of claims 2-18, wherein the first active ingredient, the pharmaceutically acceptable salt thereof, the second active ingredient, the pharmaceutically acceptable salt thereof, or any combination thereof is at least partially embedded in the first capsule, the second capsule or both.

20. The pharmaceutical or dietary supplement composition of any one of claims 3-19, wherein at least a portion of the excipient and the plurality of spray dried particles are admixed in a substantially homogenous mixture in unit dose form.

21. The pharmaceutical or dietary supplement composition of any one of claims 3-19, wherein at least a portion of the excipient and the second active ingredient, or a pharmaceutically acceptable salt thereof are admixed in a substantially homogenous mixture in unit dose form.

22. The pharmaceutical or dietary supplement composition of any one of claims 1-21, further comprising adding a capsule band to the first capsule, the second capsule, or both wherein the capsule band at least partially seals the capsule.

23. The pharmaceutical or dietary supplement composition of any one of claims 2-22, wherein the portion of the first capsule not containing the particles comprising the first active ingredient, the pharmaceutically acceptable salt thereof, the second active ingredient, the pharmaceutically acceptable salt thereof, or any combination thereof is at least partially filled with an inert gas.

24. The pharmaceutical or dietary supplement composition of any one of claims 1-23, wherein a space exists between the first capsule and the second capsule, and wherein the space is at least in part filled with an inert gas.

25. The pharmaceutical or dietary supplement composition of claim 23 or 24, wherein the inert gas comprises nitrogen, carbon dioxide, helium, or any combination thereof.

26. The pharmaceutical or dietary supplement composition of any one of claims 3-25, wherein a weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 1000:1 (w/w).

27. The pharmaceutical or dietary supplement composition of claim 26, wherein the weight to weight ratio of: a) the excipient and b) the first active ingredient, the pharmaceutically acceptable salt thereof, the second active ingredient, the pharmaceutically acceptable salt thereof, or any combination thereof ranges from about 1:1 (w/w) to about 10:1 (w/w).

28. The pharmaceutical or dietary supplement composition of any one of claims 23-27, wherein a content of the first capsule, the second capsule, or both comprise less than about 10% water by weight or wherein a total content of all gases in the capsule is less than about 10% water by weight.

29. The pharmaceutical or dietary supplement composition of any one of claims 1-28, wherein the first capsule, the second capsule, or both comprise a hydroxypropyl methylcellulose (HPMC).

30. The pharmaceutical or dietary supplement composition of any one of claims 1-29, wherein the first capsule is size: 000, 00, 0, 1, 2, 3, 4, or 5.

31. The pharmaceutical or dietary supplement composition of claim 30, wherein the first capsule is size: 1, 2, 3, 4, or 5.

32. The pharmaceutical or dietary supplement composition of any one of claims 1-29, wherein the second capsule is size: 000, 00, 0, 1, 2, 3, or 4.

33. The pharmaceutical or dietary supplement composition of claim 32, wherein the second capsule is size: 000, 00, 0, or 1.

34. The pharmaceutical or dietary supplement composition of any one of claims 1-33, wherein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

35. The pharmaceutical or dietary supplement composition of any one of claims 2-34, wherein in a human clinical trial or human safety trial, when ingested, operates mechanistically such that in at least a portion of the humans in the clinical trial or the safety trial, at least a portion of the second active ingredient is released earlier in time than the particles comprising the first active ingredient.

36. The pharmaceutical or dietary supplement composition of any one of claims 3-35, wherein the excipient comprises honey, a glycerin, an oil, a long chain sugar, a polyethylene glycol, a corn syrup, a maltodextrin syrup, a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

37. The pharmaceutical or dietary supplement composition of claim 36, comprising the excipient, wherein the excipient comprises honey.

38. The pharmaceutical or dietary supplement composition of claim 36, comprising the excipient, wherein the excipient comprises an oil and wherein the oil comprises a food oil.

39. The pharmaceutical or dietary supplement composition of claim 38, wherein the food oil comprises an almond oil, an avocado oil, a brazil nut oil, a canola oil, a cashew oil, a chia seed oil, a cocoa butter oil, a coconut oil, a corn oil, a cottonseed oil, a flaxseed/linseed oil, a grape seed oil, a hemp seed oil, a vigna mungo oil, a mustard oil, an olive oil, a bean oil, a palm oil, a peanut oil, a pecan oil, a perilla oil, a rice bran oil, a safflower oil, a sesame oil, a soybean oil, a soybean oil, a walnut oil, a sunflower oil, a cottonseed oil, a vegetable oil or any combination thereof.

40. The pharmaceutical or dietary supplement composition of claim 36, wherein the excipient comprises a carbohydrate, wherein the carbohydrate comprises a lactose, a microcrystalline cellulose, a cellulose, a mannitol, a sorbitol, a starch, a starch a glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof.

41. The pharmaceutical or dietary supplement composition of any one of claims 1-40, wherein the first active ingredient is in the form of a liquid in unit dose form.

42. The pharmaceutical or dietary supplement composition of claim 41, wherein the liquid is an oil.

43. The pharmaceutical or dietary supplement composition of claim 36, comprising the excipient wherein the excipient comprises a glycerin.

44. The pharmaceutical or dietary supplement composition of any one of claims 2-43 wherein the second active ingredient or the pharmaceutically acceptable salt thereof comprises a cannabinoid or a pharmaceutically acceptable salt thereof in unit dose form.

45. The pharmaceutical or dietary supplement composition of claim 1 or 44, wherein the cannabinoid comprises a tetrahydrocannabinol (THC), a tetrahydrocannabinol Delta-8, a tetrahydrocannabinol Delta-9, a tetrahydrocannabinol Delta-11, a tetrahydrocannabinol Delta-13, a tetrahydrocannabivarin (THCV), a tetrahydrocannabinol THCA, a full spectrum THC, or a pharmaceutically acceptable salt thereof in unit dose form.

46. The pharmaceutical or dietary supplement composition of claim 1 or 44, wherein the cannabinoid comprises a cannabichromene (CBC), a cannabichromevarin (CBCV), a cannabidiol (CBD), a cannabidivarin (CBDV), a cannabigerol (CBG), a cannabigerivarin (CBGV), a cannabinol (CBN), a cannabivarin (CBV), a THC Delta-8, or a pharmaceutically acceptable salt thereof in unit dose form.

47. The pharmaceutical or dietary supplement composition of claim 46, wherein the cannabichromene (CBC), the cannabichromevarin (CBCV), the cannabidiol (CBD), the cannabidivarin (CBDV), the cannabigerol (CBG), the cannabigerivarin (CBGV), the cannabinol (CBN), the cannabivarin (CBV), the THC Delta-8, or the pharmaceutically acceptable salt thereof in unit dose form is derived from hemp.

48. The pharmaceutical or dietary supplement composition of any one of claims 1-47, wherein the first active ingredient or the pharmaceutically acceptable salt thereof in unit dose form is present in an amount ranging from about 1 mg to about 200 mg, or from about 2 mg to about 50 mg.

49. The pharmaceutical or dietary supplement composition of any one of claims 2-48, wherein the second active ingredient or the pharmaceutically acceptable salt thereof in unit dose form is present in an amount ranging from about 1 mg to about 200 mg, or from about 2 mg to about 50 mg.

50. The pharmaceutical or dietary supplement composition of any one of claims 1-49, further comprising a further active ingredient or a pharmaceutically acceptable salt thereof in unit dose form.

51. The pharmaceutical or dietary supplement composition of claim 50, wherein the further active ingredient comprises a cannabinoid, pharmaceutically acceptable salt thereof, or both in unit dose form.

52. The pharmaceutical or dietary supplement composition of claim 51, wherein the further active ingredient comprises the cannabinoid, wherein the cannabinoid comprises a cannabichromene (CBC), a cannabichromevarin (CBCV), a cannabidiol (CBD), a cannabidivarin (CBDV), a cannabigerol (CBG), a cannabigerivarin (CBGV), a cannabinol (CBN), a cannabivarin (CBV), a THC Delta-8, a tetrahydrocannabinol (THC), a tetrahydrocannabinol Delta-8, a tetrahydrocannabinol Delta-9, a tetrahydrocannabinol Delta-11, a tetrahydrocannabinol Delta-13, a tetrahydrocannabivarin (THCV), a tetrahydrocannabinol THCA, a full spectrum THC, a pharmaceutically acceptable salt of any of these, or any combination thereof in unit dose form.

53. The pharmaceutical or dietary supplement composition of any one of claims 1-52, comprising the salt of the active ingredient, wherein the salt comprises an organic salt, an inorganic salt, or any combination thereof.

54. The pharmaceutical or dietary supplement composition of any one of claims 1-52, comprising the salt of the active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof.

55. The pharmaceutical or dietary supplement composition of any one of claims 3-54, wherein the excipient is a pharmaceutically acceptable excipient.

56. The pharmaceutical or dietary supplement composition of any one of claims 3-54, wherein the excipient is a dietary supplement acceptable excipient.

57. The pharmaceutical or dietary supplement composition of any one of claims 1-55, which is a pharmaceutical composition.

58. The pharmaceutical composition of claim 57, wherein the pharmaceutical composition is in a therapeutically effective amount to treat a disease or condition.

59. The pharmaceutical or dietary supplement composition of any one of claim 1-54 or 56, which is a dietary supplement composition.

60. The dietary supplement composition of claim 59, wherein the dietary supplement is in effective amount to maintain health of a subject.

61. A kit comprising the pharmaceutical or dietary supplement composition of any one of claims 1-60 contained at least in part in a packaging.

62. A method of making a kit, comprising at least partially packaging the pharmaceutical or dietary supplement composition of any one of claims 1-60 into a packaging.

63. A method of treating or preventing a disease or condition in a subject in need thereof, comprising treating or preventing the disease or condition by administering a therapeutically effective amount of the pharmaceutical composition of any one of claims 57-58 to the subject in need thereof.

64. The method of claim 63, wherein the administering is conducted one, two, three, or four times per day.

65. The method of any one of claims 63-64, wherein the disease or condition is selected from the group consisting of: cancer, breast cancer, melanoma, anxiety, pruritus (itching), cognitive function, Alzheimer's disease, amyotrophic lateral sclerosis, Huntington's disease, Parkinson's disease, a chronic pain, pain management, multiple sclerosis, a side effect of chemotherapy, HIV, AIDS, a neurodegenerative disorder, a behavior disorder, Tourette syndrome, cervical dystonia, sleep aide, appetite stimulant, a seizure, an epilepsy, nausea associated with chemotherapy, anorexia, spinal cord injury, sleep disorders, glaucoma, schizophrenia, epilepsy, asthma, posttraumatic stress disorder, cachexia, irritable bowel syndrome, a dependency withdrawal, psychiatric symptoms, autoimmune diseases, inflammation, sleep apnea, a headache, a migraine, opioid addiction, and any combination thereof.

66. The method of any one of claims 63-65, wherein the pharmaceutical composition is administered as needed, or for: one day, two days, three days, four days, five days, six days, a week, two weeks, three weeks, a month, two months, three months, four months, five months, six months, seven months, eight months, nine months, ten months, eleven months, a year, or chronically.

67. The method of any one of claims 63-66, wherein an amount of the active ingredient or the pharmaceutically acceptable salt thereof in the unit dose ranges from about 1 mg to about 200 mg, or from about 2 mg to about 50 mg.

68. The method of any one of claims 63-67, wherein a second therapeutic or a pharmaceutically acceptable salt thereof is administered.

69. The method of claim 68, wherein the second therapeutic or a pharmaceutically acceptable salt thereof is administered concurrently.

70. The method of claim 69, wherein the second therapeutic or the pharmaceutically acceptable salt thereof is comprised in the pharmaceutical formulation.

71. The method of claim 71, wherein the wherein the second therapeutic or the pharmaceutically acceptable salt thereof is not comprised in the pharmaceutical formulation.

72. The method of claim 68, wherein the second therapeutic or the pharmaceutically acceptable salt thereof is administered consecutively.

73. The method of any one of claims 63-72, wherein the subject is diagnosed with the disease or condition.

74. The method of claim 73, wherein the diagnosing comprises employing an in vitro diagnostic.

75. The method of claim 74, wherein the in vitro diagnostic is a companion diagnostic.

76. The method of any one of claims 63-75, wherein the administering is oral administration, rectal administration, or any combination thereof.

77. The method of claim 63, wherein the subject is a non-human animal.

78. The method of claim 77, wherein the non-human animal is a cat, a dog, a horse, a rodent, a cow, a pig, a bird, a goat, a sheep, or a reptile.

79. The method of claim 63, wherein the subject is a human.

80. The method of claim 79, wherein the subject is born as a man.

81. The method of claim 79, wherein the subject is born as a woman.

82. The method of any one of claims 79-81, wherein the subject is over 18 years of age.

83. The method of any one of claims 79-81, wherein the subject is under 18 years of age.

84. The method of any one of claim 69 or 72, wherein the second therapeutic or the pharmaceutically acceptable salt thereof is administered orally, intranasally, intraocularly, anally, by injection, intravenously, intramuscularly, subcutaneously, intraperitoneally, trans dermally, or any combination thereof.

85. A method of making the pharmaceutical or dietary supplement composition of any one of claims 1-60, wherein the method comprises formulating a capsule-in-capsule formulation in unit dose form wherein the formulation comprises:
i) a first active ingredient or a pharmaceutically acceptable salt thereof comprising a plurality of spray dried particles, each particle of the plurality of spray dried particles comprising a cannabinoid or a pharmaceutically acceptable salt thereof, substantially encapsulated in a coating material;
wherein the coating material comprises a hydroxypropyl methylcellulose (HPMC), a hydroxypropyl methylcellulose acetate succinate (HPMCAS), a maltodextrin, a povidone, a copovidone or any combination thereof; and
wherein the plurality of spray dried particles are at least partially surrounded by a first capsule, a second capsule, or both; and wherein the first capsule is surrounded by the second capsule.

86. The method of claim 85, wherein the pharmaceutical or dietary supplement composition further comprises a second active ingredient, or a pharmaceutically acceptable salt thereof in unit dose form.

87. The method of claim 85 or 86, wherein the pharmaceutical or dietary supplement composition further comprises an excipient.

88. The method of claim 86 or 87, wherein the second active ingredient is at least partially surrounded by the first capsule, the second capsule or both.

89. The method of any one of claims 86-88, wherein the second active ingredient in unit dose form is in the form of a liquid, a solid, or a powder.

90. The method of any one of claims 86-89, wherein the second active ingredient comprises particles, and wherein the particles are at least partially encapsulated by a coating material and wherein the particles at least partially encapsulated by the coating material are spray dried.

91. The method of claim 85 or 90, wherein at least a portion of the particles at least partially encapsulated by the coating material have a particle diameter ranging from about 50 micrometers to about 150 micrometers, as measured by a particle analyzer using laser diffraction.

92. The method of any one of claims 85-91, wherein the first capsule, the second capsule, or both comprise a capsule coating.

93. The method of claim 92, wherein the capsule coating at least partially controls active ingredient release.

94. The method of claim 92 or 93, wherein the capsule coating comprises an enteric coating, a time release coating, a pH dependent coating, a delayed release coating, an extended release coating, or a combination thereof.

95. The method of any one of claims 85-94, wherein the at least partially encapsulated by the coating material comprises dissolving the particles comprising the active ingredient or the pharmaceutically acceptable salt thereof in a solvent with the coating material.

96. The method of claim 92 or 93, wherein the capsule coating comprises an enteric coating.

97. The method of any one of claims 85-96, further comprising adding a capsule band to the first capsule, the second capsule, or both, wherein the capsule band seals the capsule.

98. The method of any one of claims 86-96, wherein the portion of the first capsule not containing the particles comprising the first active ingredient, the pharmaceutically acceptable salt thereof, the second active ingredient, the pharmaceutically acceptable salt thereof, or any combination thereof is at least partially filled with an inert gas.

99. The method of any one of claims 85-98, wherein a space exists between the first capsule and the second capsule, and wherein the space is at least in part filled with an inert gas.

100. The method of claim 98 or 99, wherein the inert gas comprises nitrogen, carbon dioxide, helium, or any combination thereof.

101. The method of any one of claims 85-100, wherein the first capsule, the second capsule, or both are containers that comprise the pharmaceutical or dietary supplement formulation.
